SEQUENCE LISTING (12 sequences)

SEQ ID NO: 1 - DNA, 1824 bp, Bos taurus
  sig_peptide (1)..(54)
  CDS (1)..(1821)
  misc_feature (55)..(72)
  mat_peptide (73)..(1821)
atgaagtgggtgacttttatttctcttctccttctcttcagctctgcttattccaggggtgtgtttcgtcgagatacacacaagagtgagattgctcatcggtttaaagatttgggagaagaacattttaaaggcctggtactgattgccttttctcagtatctccagcagtgtccatttgatgagcatgtaaaattagtgaacgaactaactgagtttgcaaaaacatgtgttgctgatgagtcccatgccggctgtgaaaagtcacttcacactctctttggagatgaattgtgtaaagttgcatcccttcgtgaaacctatggtgacatggctgactgctgtgagaaacaagagcctgaaagaaatgaatgcttcctgagccacaaagatgatagcccagacctccctaaattgaaaccagaccccaatactttgtgtgatgagtttaaggcagatgaaaagaagttttggggaaaatacctatacgaaattgctagaagacatccctacttttatgcaccagaactcctttactatgctaataaatataatggagtttttcaagaatgctgccaagctgaagataaaggtgcctgcctgctaccaaagattgaaactatgagagaaaaagtactgacttcatctgccagacagagactcaggtgtgccagtattcaaaaatttggagaaagagctttaaaagcatggtcagtagctcgcctgagccagaaatttcccaaggctgagtttgtagaagttaccaagctagtgacagatctcacaaaagtccacaaggaatgctgccatggtgacctacttgaatgcgcagatgacagggcagatcttgccaagtacatatgtgataatcaagatacaatctccagtaaactgaaggaatgctgtgataagcctttgttggaaaaatcccactgcattgctgaggtagaaaaagatgccatacctgaaaacctgcccccattaactgctgactttgctgaagataaggatgtttgcaaaaactatcaggaagcaaaagatgccttcctgggctcgtttttgtatgaatattcaagaaggcatcctgaatatgctgtctcagtgctattgagacttgccaaggaatatgaagccacactggaggaatgctgtgccaaagatgatccacatgcatgctattccacagtgtttgacaaacttaagcatcttgtggatgagcctcagaatttaatcaaacaaaactgtgaccaattcgaaaaacttggagagtatggattccaaaatgcgctcatagttcgttacaccaggaaagtaccccaagtgtcaactccaactctcgtggaggtttcaagaagcctaggaaaagtgggtactaggtgttgtacaaagccggaatcagaaagaatgtcctgtactgaagactatctgagcttgatcctgaaccggttgtgcgtgctgcatgagaagacaccagtgagtgaaaaagtcaccaagtgctgcacagagtcattggtgaacagacggccatgtttctctgctctgacacctgatgaaacatatgtacccaaagcctttgatgagaaattgttcaccttccatgcagatatatgcacacttcccgatactgagaaacaaatcaagaaacaaactgcacttgttgagctgttgaaacacaagcccaaggcaacagaggaacaactgaaaaccgtcatggagaattttgtggcttttgtagacaagtgctgtgcagctgatgacaaagaagcctgctttgctgtggagggtccaaaacttgttgtttcaactcaaacagccttagcctaa

SEQ ID NO: 2 - protein, 607 aa, Bos taurus
MKWVTFISLLLLFSSAYSRGVFRRDTHKSEIAHRFKDLGEEHFKGLVLIAFSQYLQQCPFDEHVKLVNELTEFAKTCVADESHAGCEKSLHTLFGDELCKVASLRETYGDMADCCEKQEPERNECFLSHKDDSPDLPKLKPDPNTLCDEFKADEKKFWGKYLYEIARRHPYFYAPELLYYANKYNGVFQECCQAEDKGACLLPKIETMREKVLTSSARQRLRCASIQKFGERALKAWSVARLSQKFPKAEFVEVTKLVTDLTKVHKECCHGDLLECADDRADLAKYICDNQDTISSKLKECCDKPLLEKSHCIAEVEKDAIPENLPPLTADFAEDKDVCKNYQEAKDAFLGSFLYEYSRRHPEYAVSVLLRLAKEYEATLEECCAKDDPHACYSTVFDKLKHLVDEPQNLIKQNCDQFEKLGEYGFQNALIVRYTRKVPQVSTPTLVEVSRSLGKVGTRCCTKPESERMSCTEDYLSLILNRLCVLHEKTPVSEKVTKCCTESLVNRRPCFSALTPDETYVPKAFDEKLFTFHADICTLPDTEKQIKKQTALVELLKHKPKATEEQLKTVMENFVAFVDKCCAADDKEACFAVEGPKLVVSTQTALA

SEQ ID NO: 3 - DNA, 1830 bp, Homo sapiens
  CDS (1)..(1827)
  sig_peptide (1)..(54)
  misc_feature (56)..(72)
  mat_peptide (73)..(1827)
atgaagtgggtaacctttatttcccttctttttctctttagctcggcttattccaggggtgtgtttcgtcgagatgcacacaagagtgaggttgctcatcggtttaaagatttgggagaagaaaatttcaaagccttggtgttgattgcctttgctcagtatcttcagcagtgtccatttgaagatcatgtaaaattagtgaatgaagtaactgaatttgcaaaaacatgtgttgctgatgagtcagctgaaaattgtgacaaatcacttcataccctttttggagacaaattatgcacagttgcaactcttcgtgaaacctatggtgaaatggctgactgctgtgcaaaacaagaacctgagagaaatgaatgcttcttgcaacacaaagatgacaacccaaacctcccccgattggtgagaccagaggttgatgtgatgtgcactgcttttcatgacaatgaagagacatttttgaaaaaatacttatatgaaattgccagaagacatccttacttttatgccccggaactccttttctttgctaaaaggtataaagctgcttttacagaatgttgccaagctgctgataaagctgcctgcctgttgccaaagctcgatgaacttcgggatgaagggaaggcttcgtctgccaaacagagactcaagtgtgccagtctccaaaaatttggagaaagagctttcaaagcatgggcagtagctcgcctgagccagagatttcccaaagctgagtttgcagaagtttccaagttagtgacagatcttaccaaagtccacacggaatgctgccatggagatctgcttgaatgtgctgatgacagggcggaccttgccaagtatatctgtgaaaatcaagattcgatctccagtaaactgaaggaatgctgtgaaaaacctctgttggaaaaatcccactgcattgccgaagtggaaaatgatgagatgcctgctgacttgccttcattagctgctgattttgttgaaagtaaggatgtttgcaaaaactatgctgaggcaaaggatgtcttcctgggcatgtttttgtatgaatatgcaagaaggcatcctgattactctgtcgtgctgctgctgagacttgccaagacatatgaaaccactctagagaagtgctgtgccgctgcagatcctcatgaatgctatgccaaagtgttcgatgaatttaaacctcttgtggaagagcctcagaatttaatcaaacaaaattgtgagctttttgagcagcttggagagtacaaattccagaatgcgctattagttcgttacaccaagaaagtaccccaagtgtcaactccaactcttgtagaggtctcaagaaacctaggaaaagtgggcagcaaatgttgtaaacatcctgaagcaaaaagaatgccctgtgcagaagactatctatccgtggtcctgaaccagttatgtgtgttgcatgagaaaacgccagtaagtgacagagtcaccaaatgctgcacagaatccttggtgaacaggcgaccatgcttttcagctctggaagtcgatgaaacatacgttcccaaagagtttaatgctgaaacattcaccttccatgcagatatatgcacactttctgagaaggagagacaaatcaagaaacaaactgcacttgttgagctcgtgaaacacaagcccaaggcaacaaaagagcaactgaaagctgttatggatgatttcgcagcttttgtagagaagtgctgcaaggctgacgataaggagacctgctttgccgaggagggtaaaaaacttgttgctgcaagtcaagctgccttaggcttataa

SEQ ID NO: 4 - protein, 609 aa, Homo sapiens [sequence omitted]

SEQ ID NO: 5 - DNA, 1821 bp, Artificial
  Synthetic CDS for BSA
atgaaatgggtgaccttcatatccttgctgttgctgttcagcagtgcctactctaggggcgtgttcaggagggacacacacaagagtgagattgcacatcggttcaaagacttgggagaagagcatttcaagggcttggtcctcattgcattctcccaatatcttcagcagtgcccgttcgatgagcacgtcaagttggtgaatgaactcaccgagtttgcgaagacatgcgtagcggacgagtcccatgctggctgtgagaagtccctacatacgctgttcggcgacgagctttgcaaagtggcatcgctccgagagacgtatggagatatggcagactgttgcgagaagcaggagccagaacgcaatgaatgtttcttgtcccacaaggacgacagtcccgatttgcccaagctcaagcccgaccccaacacactctgcgacgagttcaaagccgatgagaagaaattctggggaaagtatctctatgagatcgcgaggcgccatccctacttctacgcaccggaattgttgtactacgcaaataagtacaacggagtgtttcaggagtgttgccaggccgaggacaagggagcctgtctccttcccaaaatagaaacaatgagggagaaggtgcttgcgtctagcgcccggcagcgcctccgctgtgcatcgatccagaagttcggtgagcgagccctcaaagcatggtcggttgccaggctatcccaaaagttccccaaagcagaattcgtcgaggtgacaaaattggtcaccgacctcacgaaggtgcacaaggagtgttgtcatggcgaccttttggagtgtgccgatgaccgcgcagatctggcaaaatacatttgtgataaccaagacaccatctccagcaagctcaaggagtgttgtgacaagccgcttttggagaagagtcactgtatagccgaggtcgaaaaagacgcgattccggaaaacttgcctcccctcacggccgacttcgcagaggataaagatgtatgcaagaactaccaggaggcaaaggatgctttcctcggctcgttcttgtatgaatacagcaggcgccacccagaatatgctgtttcagtccttcttcgcctagcgaaagaatacgaagccaccctcgaagaatgttgtgcgaaagacgacccccacgcatgctactccactgtgttcgataagctcaagcacctcgtcgacgagcctcaaaaccttatcaagcagaactgtgatcagttcgagaaattgggagaatatggctttcagaacgccctcatagtgcgctacacccggaaggtcccccaagtctccacaccaaccctggtcgaagtatcccggtcgttgggcaaggtcggtactcgctgttgcacgaagcccgagagtgagaggatgccctgtaccgaagactacctctcgctcatcctcaataggttgtgtgtgctccatgagaagactcccgtgtcggagaaggtgaccaagtgttgtaccgaatcgctggtgaaccgcaggccatgtttctcggcgctcacacccgacgaaacgtatgtcccgaaagccttcgacgaaaaattgttcacgttccatgcggacatttgcacgctaccggatacagaaaaacagataaagaagcaaaccgccttggtcgagttgctcaagcataagcccaaggcgaccgaggaacagctcaagacggttatggagaacttcgtcgccttcgtcgataaatgttgtgcagccgacgacaaggaggcttgttttgccgtcgagggtccaaagctcgttgtcagcacccagaccgccctggcg

SEQ ID NO: 6 - protein, 607 aa, Artificial
  Synthetic CDS for BSA
MKWVTFISLLLLFSSAYSRGVFRRDTHKSEIAHRFKDLGEEHFKGLVLIAFSQYLQQCPFDEHVKLVNELTEFAKTCVADESHAGCEKSLHTLFGDELCKVASLRETYGDMADCCEKQEPERNECFLSHKDDSPDLPKLKPDPNTLCDEFKADEKKFWGKYLYEIARRHPYFYAPELLYYANKYNGVFQECCQAEDKGACLLPKIETMREKVLASSARQRLRCASIQKFGERALKAWSVARLSQKFPKAEFVEVTKLVTDLTKVHKECCHGDLLECADDRADLAKYICDNQDTISSKLKECCDKPLLEKSHCIAEVEKDAIPENLPPLTADFAEDKDVCKNYQEAKDAFLGSFLYEYSRRHPEYAVSVLLRLAKEYEATLEECCAKDDPHACYSTVFDKLKHLVDEPQNLIKQNCDQFEKLGEYGFQNALIVRYTRKVPQVSTPTLVEVSRSLGKVGTRCCTKPESERMPCTEDYLSLILNRLCVLHEKTPVSEKVTKCCTESLVNRRPCFSALTPDETYVPKAFDEKLFTFHADICTLPDTEKQIKKQTALVELLKHKPKATEEQLKTVMENFVAFVDKCCAADDKEACFAVEGPKLVVSTQTALA

SEQ ID NO: 7 - DNA, 1827 bp, Artificial
  Synthetic CDS for HSA
atgaaatgggtcaccttcatctccctcctcttcctgttttctagcgcatattcgcgaggcgtgttcaggcgtgatgcgcataagtccgaagtggcacaccgtttcaaagatctcggggaggagaacttcaaggccttggtgctcatagcattcgcacagtatcttcagcagtgtccattcgaagaccacgttaaactcgttaacgaggtcacagagttcgccaagacctgtgtagccgatgagtcggcggaaaattgtgataagagtctgcacaccctcttcggagataagctttgtacggtggcgacgctcagggaaacctatggtgagatggccgactgctgcgcgaagcaggagcccgagcgtaacgagtgcttcttgcagcacaaggatgacaaccccaacctgcctcgactcgtaaggccagaggtcgatgtcatgtgtacggcattccatgacaacgaagagaccttcttaaaaaagtacctgtacgagatcgcacgccgccacccttacttctatgcacccgagctcctattcttcgccaagcgatacaaggcagccttcaccgagtgttgtcaagcagccgacaaggcggcgtgtctattaccgaaattggatgagctcagggatgaggggaaggcgtcatccgcaaagcagaggttgaagtgtgcgtccttgcagaagttcggtgagcgcgcattcaaagcgtgggcagtggcacgcctcagccagcgtttcccaaaggctgaattcgcagaagtatcgaagctcgtgaccgatctcaccaaagtgcacaccgagtgctgtcatggtgatctgctcgagtgtgcagacgatagggccgatctcgccaaatacatttgtgaaaaccaggactcaatctcgagtaagctaaaagagtgttgtgagaaaccgctgctggagaaatcccattgcatagccgaggtggaaaacgacgagatgccggccgatcttccaagcctggctgccgatttcgtggaatccaaggatgtttgtaagaactacgccgaggcgaaggacgtttttctcggaatgtttctgtacgagtatgccaggaggcatcccgattacagcgtggtactcctcctgcggctcgcaaaaacgtacgaaacaacactcgaaaagtgctgcgcggccgccgaccctcatgagtgttacgcaaaggtgttcgatgagttcaagccccttgtagaggagccgcagaacctcatcaagcaaaactgtgagcttttcgaacagttgggcgaatataagttccaaaacgcactccttgtcaggtacactaagaaagttccccaggtatcgacacccactcttgtcgaggtcagccgaaacctagggaaagttggttccaaatgttgtaagcatcccgaggctaaacgcatgccgtgtgcagaggactacttgtcggtggtcctcaaccaactgtgcgtcctccatgagaagacaccagtgtccgaccgtgtgaccaagtgttgtacagaatccctcgtcaaccggcgtccatgcttcagcgcccttgaagtcgatgaaacttacgttcccaaagagttcaatgccgaaactttcacattccacgcggacatttgtactctctcggaaaaggaacgacagattaaaaaacagacagccttggtcgagcttgtcaaacacaagcccaaggcgactaaagaacagctgaaagcagtaatggatgatttcgccgccttcgtcgagaagtgttgtaaagccgacgacaaggagacctgttttgcagaggagggcaagaagctggtggctgcctcgcaggccgctttgggtttg

SEQ ID NO: 8 - protein, 609 aa, Artificial [sequence omitted]

SEQ ID NO: 9 - DNA, 39 bp, Artificial
  Primer
atggacggatccacaatgaagtgggtaacctttatttcc

SEQ ID NO: 10 - DNA, 45 bp, Artificial
  Primer
atggacccgcggctcgagttataagcctaaggcagcttgacttgc

SEQ ID NO: 11 - DNA, 40 bp, Artificial
  Primer
gactcgggatccacaatgaagtgggtgacttttatttctc

SEQ ID NO: 12 - DNA, 42 bp, Artificial
  Primer
gactcgccgcggctcgagttaggctaaggctgtttgagttga

(19) United States
(12) Patent Application Publication
(10) Pub. No.: US 2009/0280534 A1
(43) Pub. Date: Nov. 12, 2009

(54) Recombinant Production of Serum Albumin

(75) Inventors: Bjarke Christensen, Lyngby (DK); Carsten Hjort, Vaerlose (DK); Thomas Agersten Poulsen, Ballerup (DK); Poul Bach, Birkeroed (DK)

Correspondence Address: NOVOZYMES NORTH AMERICA, INC., 500 FIFTH AVENUE, SUITE 1600, NEW YORK, NY, 10110, US

(73) Assignee: Novozymes A/S, Bagsvaerd (DK)

(21) Appl. No.: 11/721,813

(22) PCT Filed: Dec. 22, 2005
(86) PCT No.: PCT/DK05/00818

§ 371 Date: Jun. 15, 2007

Related U.S. Application Data
(60) Provisional application No. 60/662,319, filed on Mar. 16, 2005.
(60) Provisional application No. 60/638,583, filed on Dec. 22, 2004.

(30) Foreign Application Priority Data
Dec. 22, 2004 (DK) PA 2004 01974
Mar. 1, 2005 (DK) PA 2005 00310

(51) Int. Cl. C12P 21/02; C12N 15/11

(52) U.S. Cl. 435/69.6; 536/23.5; 530/363

(57) ABSTRACT

The present invention relates to modified nucleotide sequences encoding serum albumin, wherein the mRNA sequence has been codon optimized for expression in a filamentous host cell. Furthermore the present invention relates to serum albumin produced in filamentous fungi or loaded by contacting serum albumin with an extract of a filamentous fungi culture. In an-other aspect the present invention relates to the use of said serum albumin in serum free cell culture media and also to a method of drying and agglomerating serum albumin thereby im-proving wettability and dispersability.

### FIELD OF THE INVENTION

The present invention relates to methods for recombinant expression of serum albumin in a filamentous fungal host organism, to modified nucleic acid sequences encoding bovine and human serum albumin proteins (BSA and HSA, respectively), to a loaded serum albumin product, to a use of the loaded serum albumin, and to a process for the preparation of a dry particle as well as a dry particle comprising serum albumin.

### BACKGROUND OF THE INVENTION

Serum albumin, especially bovine serum albumin (BSA), is one of the most widely used proteins in the field of molecular biology and industry.

Serum albumin like e.g. BSA or HSA has previously been obtained by blood fractionation. This way of providing serum albumin suffers from the drawback of possible contamination with pathogens. In the case of BSA, the presence of prions thought to be responsible for madcow disease (BSE) is in particular a problem associated with the production of BSA by blood fractionation. For HSA there is a risk of possible contamination by hepatitis and immunodeficiency viruses like HIV, when HSA is produced by blood fractionation.

Alternative methods for providing serum albumin which is free of contaminants and which can be produced in economical yields are therefore desirable.

Recombinant expression of BSA/HSA would solve the problem of contamination described above. Also such recombinant serum albumin would be better defined in terms of other contaminants originating from blood plasma like e.g. blood plasma proteins and peptides (globulins). For many therapeutic uses and for obtaining more defined cell culture media, especially serum free media, recombinant serum albumin would be preferable.

Recombinant expression of proteins is however not always straight forward and obtaining sufficient yields is hard to predict for a given protein in a given expression host organism. The recombinant expression of albumin in yeast has been reported in WO 00/44772, EP 0683233 A2, and U.S. Pat. No. 5,612,196.

After expression in a host organism or purified from serum, albumin can be processed in order to improve further downstream applications. It is well known in the art to spray dry serum albumin (SA) to obtain an agglomerate free powder. WO 04/058156 discloses methods providing stable powder particles containing bioactive materials. The methods include, e.g., high pressure spraying of the bioactive materials in solution or suspension, with viscosity enhancing agents and/or surfactants.

WO 03/087335 discloses methods and compositions to preserve bioactive materials. The methods include high-pressure gas spraying and/or near supercritical spraying of formulations followed by drying in a stream of conditioned gas to form stable powder particles containing bioactive materials.

Serum albumin (SA) is often used in applications were it is of great importance that the SA is sterile. The problem has been how to obtain a sterile SA product and keeping the SA sterile upon storage and transport. A known method to use is freeze drying, however, there are some drawbacks in using freeze drying. During freeze drying some SA monomers reacts into dimers and trimers. This starting polymerization is fundamentally changing the properties of SA, in a way that makes the SA unusable for desired applications. During simple spray drying primary particles with a very low particle size are obtained. Said primary particles can not readily disperse in liquids. The primary particles stick together and make a gel like substance when added to a liquid. It would be desirable to obtain a SA product which readily disperses when added to a liquid and is thus fast dissolving.

The present invention provides a method for the efficient recombinant expression of serum albumin, in a filamentous fungal host suitable for industrial production. Further more the present invention provides a serum albumin product which can replace albumin produced from serum in all previously described uses and which in addition has several advantageous properties when used in serum free cell culture medium. In an additional aspect the present invention provides a method for improving the solubility of albumin.

### SUMMARY OF THE INVENTION

A first aspect of the present invention relates to a method for recombinant expression of a wild type serum albumin polypeptide in a filamentous fungal host organism comprising expressing a modified nucleic acid sequence encoding a wild type serum albumin polypeptide in a filamentous fungal host organism, wherein the modified nucleic acid sequence differs in at least one codon from each wild type nucleic acid sequence encoding said wild type serum albumin polypeptide.

In a second aspect the present invention relates to a method for recombinant expression of wild type serum albumin in a filamentous fungal host organism, comprising the steps:

i) providing a nucleic acid sequence encoding wild type serum albumin said nucleic add sequence comprising at least one modified codon, wherein the modification does not change the amino acid encoded by said codon and the nucleic acid sequence of said codon is different compared to the corresponding codon in the nucleic acid sequence encoding the wild type gene;
ii) expressing the modified nucleic acid sequence in the filamentous fungal host.

A third aspect of the present invention relates to a modified nucleic acid sequence encoding a wild type bovine serum albumin polypeptide and capable of expression in a filamentous fungal host organism, wherein said modified nucleic acid sequence differs in at least one codon from each wild type nucleic acid sequence encoding said wild type bovine serum albumin polypeptide.

A fourth aspect of the present invention relates to a modified nucleic acid sequence encoding a wild type human serum albumin polypeptide and capable of expression in a filamentous fungal host organism, wherein said modified nucleic acid sequence differs in at least one codon from each wild type nucleic acid sequence encoding said wild type human serum albumin polypeptide.

A fifth aspect of the present invention relates to a modified nucleic acid sequence encoding the wild type BSA protein and capable of expression in a filamentous fungal host organism, which modified nucleic acid sequence is obtainable by:

i) providing the wild type nucleic acid sequence encoding BSA;
ii) modifying at least one codon, wherein the modification does not change the amino acid encoded by said codon and the nucleic acid sequence of said codon is different compared to the corresponding codon in the wild type gene.

A sixth aspect of the present invention relates to a modified nucleic acid sequence encoding the wild type HSA protein and capable of expression in a filamentous fungal host organism, which modified nucleic acid sequence is obtainable by:

i) providing the wild type nucleic acid sequence encoding HSA;
ii) modifying at least one codon, wherein the modification does not change the amino acid encoded by said codon and the nucleic acid sequence of said codon is different compared to the corresponding codon in the wild type gene.

In a seventh aspect the present invention relates to a modified nucleic acid sequence encoding the wild type BSA protein and capable of expression in a filamentous fungal host organism, wherein:

a) the modified sequence has at least 77% identity with SEQ ID NO: 5; or
b) the modified sequence hybridizes under high stringency conditions with a polynucleotide probe consisting of the complementary strand of nucleotides 1 to 1821 of SEQ ID NO: 5.

In an eight aspect the present invention relates to a modified nucleic acid sequence encoding the wild type HSA protein and capable of expression in a filamentous fungal host organism, wherein:

a) the modified sequence has at least 77% identity with SEQ ID NO: 7; or
b) the modified sequence hybridizes under high stringency conditions with a polynucleotide probe consisting of the complementary strand of nucleotides 1 to 1827 of SEQ ID NO: 7.

In a ninth aspect the present invention relates to a loaded serum albumin obtainable by:

i) recombinant expression of a nucleic acid sequence encoding the serum albumin in a filamentous fungal host cell; and/or
ii) loading the serum albumin by contacting said serum albumin with a cell extract derived from filamentous fungal cells.

A tenth aspect of the invention relates to a use of the loaded serum albumin according to the invention, in a cell culture medium.

In a further aspect the invention relates to a dry particle comprising serum albumin, wherein said particle is dried and agglomerated.

Furthermore the present invention relates to a method of producing a dried and agglomerated product comprising serum albumin, the method comprising the steps of:

a) drying a liquid composition comprising serum albumin; and
b) agglomerating the dried product.

In another aspect the present invention relates to a product comprising serum albumin, wherein said product is dried and have a particle size of at least 50 microns.

### BRIEF DESCRIPTION OF THE DRAWINGS

The invention is further illustrated by the accompanying drawings in which:

FIG. 1 shows a restriction map of the Aspergillus expression plasmid pCaHj620 comprising a synthetic HSA gene according to the invention. The NA2 promoter is the modified neutral amylase promoter from Aspergillus niger. Tamg is the amyloglycosidase terminator from Aspergillus niger. HSA is the synthetic human serum albumin gene. amdS is the acetamidase gene from Aspergillus nidulans. pUC 19 is a fragment of the Escherichia coli vector pUC19. Pura3, URA3 and Tura3 are the promoter region ORF and terminator sequence of the Saccharomyces cerevisiae URA3 gene.

FIG. 2 shows a restriction map of the Aspergillus expression plasmid pCaHj623 comprising a synthetic BSA gene according to the invention. NA2 promoter is the modified neutral amylase promoter from Aspergillus niger. Tamg is the amyloglycosidase terminator from Aspergillus niger. BSA is the synthetic bovine serum albumin gene. amdS is the acetamidase gene from Aspergillus nidulans. pUC19 is a fragment of the Escherichia coli vector pUC19. Pura3, URA3 and Tura3 are the promoter region ORF and terminator sequence of the Saccharomyces cerevisiae URA3 gene.

FIG. 3 shows the HPLC analysis of the spray dried powder vs. the r-HSA concentrates used. The results show that no significant polymerisation takes place during spray drying as the elution profiles are essential identical.

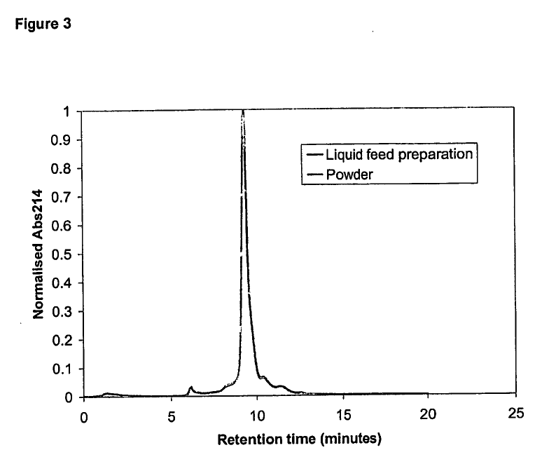

FIG. 4 shows the effect of replacing natural HSA purified from serum in cell culture medium with rHSA expressed in A. oryzae. The figure shows cell growth, measured by incorporation of radioactively labelled thymidine, as a function of the concentration of HSA in the culture medium in μg/ml.

FIG. 5 shows the effect of replacing natural HSA purified from serum in cell culture medium with loaded HSA, wherein the loaded HSA was obtained by incubating a fatty acid free commercial HSA product with a cell lysate derived from a culture of A. oryzae. The figure shows cell growth, measured by incorporation of radioactively labelled thymidine, as a function of the concentration of HSA in the culture medium in μg/ml.

### DETAILED DESCRIPTION OF THE INVENTION

### Serum Albumin

Serum albumin according to the present invention comprises a class of small globular proteins found in blood, synovial fluid, milk and other mammalian secretions. Serum albumin is the protein of the highest concentration in plasma, and transports many small molecules in the blood (e.g. bilirubin, calcium, progesterone and drugs). Serum albumin belongs to the ALB/AFP/VDB family.

In the context of the present invention the term “wild type serum albumin polypeptide” is to be understood as a naturally occurring mature serum albumin polypeptide. For most genes different alleles of the same gene exist naturally, i.e. different sequences of a gene that occupy a given genetic locus on a chromosome exist naturally. Some of these differences are only present at the nucleic acid level while others are also present at the amino acid level. In the context of the present invention the term “wild type serum albumin polypeptide” is intended to encompass all the naturally occurring mature serum albumin polypeptides. For example “wild type human serum albumin polypeptide” and “wild type bovine serum albumin polypeptide” encompass all naturally occurring mature polypeptides obtainable from human and bovine, respectively. In the context of the present invention the term “mature” is to be understood as the mature part of the translated amino acid sequence. For some polypeptides the amino acid sequence which is translated from the mRNA includes besides the mature polypeptide also a signal peptide and/or a pro-peptide. Thus the signal peptide and the pro-peptide are not regarded as being part of the mature peptide.

In a particular embodiment of the present invention the serum albumin may be human or bovine serum albumin, i.e. HSA or BSA. Several variations in the amino acid sequence of the naturally occurring BSA or HSA proteins have been reported in the literature (Meloun et al., FEBS Lett. 58:134-137 (1975); Lawn et al., Nucleic Acid Res. 9:6103-6114 (1981); Dugaiczyk et al., Proc. Natl. Acad. Sci. U.S.A. 79:71-75 (1982)).

In the context of the present invention a wild type HSA polypeptide is to be understood as to include any of the mature polypeptides described in Swissprot entry P02768, and a wild type BSA polypeptide is to be understood as to include any of the mature polypeptides described in Swissprot entry P02769.

In particular the wild type BSA polypeptide may be the mature peptide shown in SEQ ID NO: 1 or 2, i.e. amino acids 1-583 of SEQ ID NO: 1 or SEQ ID NO: 2.

In particular the wild type HSA polypeptide may be the mature peptide shown in SEQ ID NO: 3 or 4, i.e. amino acids 1-585 of SEQ ID NO: 3 or SEQ ID NO: 4.

The term “wild type nucleic acid sequence encoding a wild type serum albumin polypeptide” is in the context of the present invention to be understood as a naturally occurring nucleic acid sequence encoding the translated sequence of a wild type serum albumin polypeptide, i.e. besides the nucleic acid sequence encoding the mature polypeptide it also includes the nucleic acid sequence encoding e.g. the signal peptide and the pro-peptide. The translated sequence is also known as CDS. As described above different alleles of a particular gene often exist and “wild type nucleic acid sequence encoding a wild type serum albumin polypeptide” is in the context of the present invention intended to encompass all such naturally occurring nucleic acid sequences encoding a particular wild type serum albumin polypeptide.

In the context of the present invention a wild type nucleic acid sequence encoding a wild type human serum albumin polypeptide is to be understood as to include any of the CDS sequences described in the references which are included in Swissprot entry P02768, and a wild type nucleic acid sequence encoding a wild type bovine serum albumin polypeptide is to be understood as to include any of the CDS sequences described in the references which are included in Swissprot entry P02769.

In particular the wild type nucleic acid sequence encoding a wild type bovine serum albumin polypeptide may be the CDS shown in SEQ ID NO: 1, i.e. nucleotide 1-1821 of SEQ ID NO: 1.

In particular the wild type nucleic acid sequence encoding a wild type human serum albumin polypeptide may be the CDS shown in SEQ ID NO: 3, i.e. nucleotides 1-1827 of SEQ ID NO: 3.

### Method of the Invention

Bovine serum albumin (BSA) is a widely used protein within the field of molecular biology and therefore a commercially interesting product. Human serum albumin (HSA) is especially used in therapeutic applications but can also replace BSA in most applications were BSA is commonly used.

The known way of producing BSA or HSA as described above involves blood fractionation which in turn may lead to possible contamination with pathogens.

According to the present invention the BSA or HSA is produced as a recombinant protein in a filamentous fungal host cell, which solves the problem of contamination as well as observed problems of expressing the wild type genes in filamentous fungi.

Recombinant expression of proteins is not always straight forward and it is hard to predict whether the desired product can in fact be produced in a particular production host organism and whether product yields will be sufficient for establishing an economical production.

In respect of BSA and HSA a first attempted was made to express the proteins from a normal wild type nucleic acid sequences (the nucleic acid sequences shown in SEQ ID NO: 1 and 3, respectively) in a filamentous fungal. However, no expression could be detected.

Several possibilities or combination of possibilities exist for explaining the lack of expression observed in filamentous fungal hosts. In general expression of a secreted and correctly processed albumin in a filamentous fungus involves a number of steps any of which could be a limiting step.

First the inserted albumin gene is transcribed to hnRNA. Then the hnRNA is trans-ported from the nucleus to the cytosol, and during this process it is maturated to mRNA. Generally a mRNA pool is established in the cytosol in order to sustain translation. The mRNA is then translated to a protein precursor, and this precursor is subsequently secreted to the endoplasmatic reticulum (ER) either co-translationally or post-translationally. Upon translocation into the ER the secretion signal peptide is cleaved of by a signal peptidase, and the resulting protein is folded in the ER. Secretion of the protein to the golgi apparatus follows when proper folding has been recognized by the cell. Here the propeptide will be cleaved to release the mature albumin. Thus numerous possibilities exist for preventing sufficient expression of a gene sequence in a given host organism.

In order to provide efficient expression of a polynucleotide sequence encoding a desired protein the translation process has to be efficient. One object of the present invention is therefore to optimize the mRNA sequence encoding the serum albumin protein in order to obtain sufficient expression in a filamentous fungal host cell.

In one embodiment the present invention relates to a method for recombinant expression of a wild type serum albumin polypeptide in a filamentous fungal host organism comprising expressing a modified nucleic acid sequence encoding a wild type serum albumin polypeptide in a filamentous fungal host organism, wherein the modified nucleic acid sequence differs in at least one codon from each wild type nucleic acid sequence encoding said wild type serum albumin polypeptide.

The modified nucleic acid sequence may be obtained by a) providing a wild type nucleic acid sequence encoding a wild type serum albumin polypeptide and b) modifying at least one codon of said nucleic acid sequence so that the modified nucleic acid sequence differs in at least one codon from each wild type nucleic acid sequence encoding said wild type serum albumin polypeptide. Methods for modifying nucleic acid sequences are well known to a person skilled in the art. In a particular embodiment said modification does not change the identity of the amino acid encoded by said codon.

Thus in another aspect the object of the present invention is provided by a method for recombinant expression of wild type serum albumin in a filamentous fungal host organism, comprising the steps:

i) providing a nucleic acid sequence encoding wild type serum albumin said nucleic acid sequence comprising at least one modified codon, wherein the modification does not change the amino acid encoded by said codon and the nucleic acid sequence of said codon is different compared to the corresponding codon in the nucleic acid sequence encoding the wild type gene;
ii) expressing the modified nucleic acid sequence in the filamentous fungal host.

The starting nucleic acid sequence to be modified according to this embodiment is a wild type nucleic acid sequence encoding the serum albumin of interest.

Modifications according to the invention, comprises any modification of the base triplet and in a particular embodiment they comprise any modification which does not change the identity of the amino acid encoded by said codon, i.e. the amino acid encoded by the original codon and the modified codon is the same. In most cases the modification will be at the third position, however, in a few cases the modification may also be at the first or the second position. How to modify a codon also without modifying the resulting amino acid is known to the skilled person.

For both of the above embodiments the number of codon which should differ or the number of modifications needed in order to obtain sufficient expression may vary. Thus according to a further embodiment of the invention the modified nucleic acid sequence differs in at least 2 codons from each wild type nucleic acid sequence encoding said wild type serum albumin polypeptide or at least 2 codons have been modified, particularly at least 3 codons, more particularly at least 5 codons, more particularly at least 10 codons, more particularly at least 15 codons, even more particularly at least 25 codons.

It has furthermore been found, that by changing the codon usage of the wild type nucleic acid sequence to be selected among the codons preferably used by the filamentous fungus used as a host, the expression of BSA and HSA is now possible. Such codons are said to be “optimized” for expression.

In one attempt to express BSA and HSA as recombinant proteins expressed from the wild type sequence in fungal systems the level of expression was very low, about 1 mg/l using the native genes and Aspergillus as the host cell.

Due to the degeneracy of the genetic code and the preference of certain preferred codons in particular organisms/cells the expression level of a protein in a given host cell can in some instances be improved by optimizing the codon usage. In the present case the yields of BSA and HSA were increased dramatic when wild type nucleic acid sequences encoding BSA and HSA were optimized by, among other things, codon optimization and expressed in Aspergillus.

In the present invention “codon optimized” means that due to the degeneracy of the genetic code more than one triplet codon can be used for each amino acid. Some codons will be preferred in a particular organism and by changing the codon usage in a wild type gene to a codon usage preferred in a particular expression host organism the codons are said to be optimized. Codon optimization can be performed e.g. as described in Gustafsson et al., 2004, (Trends in Biotechnology vol. 22 (7); Codon bias and heterologous protein expression), and U.S. Pat. No. 6,818,752.

Codon optimization may be based on the average codon usage for the host organism or it can be based on the codon usage for a particular gene which is know to be expressed in high amounts in a particular host cell.

In one embodiment of the invention the serum albumin protein is encoded by a modified nucleic acid sequence codon optimized in at least 10% of the codons, more particularly at least 20%, or at least 30%, or at least 40%, or particularly at least 50%, more particularly at least 60%, and more particularly at least 75%. Thus the modified nucleic acid sequence may differ in at least 10% of the codons from each wild type nucleic acid sequence encoding said wild type serum albumin polypeptide, more particularly in at least 20%, or in at least 30%, or in at least 40%, or particularly in at least 50%, more particularly in at least 60%, and more particularly in at least 75%. In particular said codons may differ because they have been codon optimized as compared with a wild type nucleic acid sequence encoding a wild type serum albumin polypeptide.

Particularly 100% of the nucleic acid sequence has been codon optimized to match the preferred codons used in filamentous fungi.

In a particular embodiment the codon optimization is based on the codon usage of alpha amylase from Aspergillus oryzae, also known as Fungamyl™ (PCT/DK 2004/000558; SEQ ID NO: 2), which is a protein known to be expressed in high levels in filamentous fungi. In the present context an expression level corresponding to at least 20% of the total amount of secreted protein constitutes the protein of interest is considered a high level of expression. Particularly at least 30%, more particularly at least 40%, even more particularly at least 50%.

In a particular embodiment, the modified nucleic acid sequence encoding BSA or HSA is selected from the group consisting of SEQ ID NO: 5 (BSA) and SEQ ID NO: 7 (HSA).

In practice the optimization according to the invention comprises the steps:

i) the nucleic acid sequence encoding the serum albumin is codon optimized as explained in more detail below;
ii) check the resulting modified sequence for a balanced GC-content (approximately 45-55%); and
iii) check or edit the resulting modified sequence from step ii) as explained below.

### Codon Optimization Protocol:

The codon usage of a single gene, a number of genes or a whole genome can be calculated with the program cusp from the EMBOSS-package (http://www.rfcgr.mrc.ac.uk/Software/EMBOSS/).

The starting point for the optimization is the amino acid sequence of the protein or a nucleic acid sequence coding for the protein together with a codon-table. By a codon-optimized gene, we understand a nucleic acid sequence, encoding a given protein sequence and with the codon statistics given by a codon table.

The codon statistics referred to is a column in the codon-table called “Fract” in the output from cusp-program and which describes the fraction of a given codon among the other synonymous codons. We call this the local score. If for instance 80% of the codons coding for F is TTC and 20% of the codons coding for F are TTT, then the codon TTC has a local score of 0.8 and TTT has a local score of 0.2.

The codons in the codon table are re-ordered first encoding amino acid (e.g. alphabetically) and then increasingly by the score. In the example above, ordering the codons for F as TTT, TTC. Cumulated scores for the codons are then generated by adding the scores in order. In the example above TTT has a cumulated score of 0.2 and TTC has a cumulated score of 1. The most used codon will always have a cumulated score of 1.

In order to generate a codon optimized gene the following is performed. For each position in the amino acid sequence, a random number between 0 and 1 is generated. This is done by the random-number generator on the computer system on which the program runs. The first codon is chosen as the codon with a cumulated score greater than or equal to the generated random number. If, in the example above, a particular position in the gene is “F” and the random number generator gives 0.5, TTC is chosen as codon.

The strategy for avoiding introns is to make sure that there are no branch points. This was done by making sure that the consensus sequence for branch-point in Aspergillus oryzae: CT[AG]A[CT] was not present in the sequence. The inventors of the present invention have also found that the sequence [AG]CT[AG]A[AG] may be recognised as a branch points in introns. Thus in a particular embodiment of the present invention such sequences may also be modified or be removed according to a method of the present invention. This was done in a post processing step, where the sequence was scanned for the presence of this motif, and each occurrence was removed by changing codons in the motif to synonymous codons, choosing codons with the best local score first.

A codon table showing the codon usage of the alpha amylase from Aspergillus oryzae is given below.

TABLE 1
Codon usage for the A. oryzae alpha amylase
(CUSP codon usage file)

| Codon | Amino acid | Fract | /1000 | Number |
|---|---|---|---|---|
| GCA | A | 0.286 | 24.000 | 12 |
| GCC | A | 0.357 | 30.000 | 15 |
| GCG | A | 0.238 | 20.000 | 10 |
| GCT | A | 0.119 | 10.000 | 5 |
| TGC | C | 0.222 | 4.000 | 2 |
| TGT | C | 0.778 | 14.000 | 7 |
| GAC | D | 0.524 | 44.000 | 22 |
| GAT | D | 0.476 | 40.000 | 20 |
| GAA | E | 0.417 | 10.000 | 5 |
| GAG | E | 0.583 | 14.000 | 7 |
| TTC | F | 0.800 | 24.000 | 12 |
| TTT | F | 0.200 | 6.000 | 3 |
| GGA | G | 0.233 | 20.000 | 10 |
| GGC | G | 0.419 | 36.000 | 18 |
| GGG | G | 0.116 | 10.000 | 5 |
| GGT | G | 0.233 | 20.000 | 10 |
| CAC | H | 0.571 | 8.000 | 4 |
| CAT | H | 0.429 | 6.000 | 3 |
| ATA | I | 0.071 | 4.000 | 2 |
| ATC | I | 0.679 | 38.000 | 19 |
| ATT | I | 0.250 | 14.000 | 7 |
| AAA | K | 0.350 | 14.000 | 7 |
| AAG | K | 0.650 | 26.000 | 13 |
| CTA | L | 0.081 | 6.000 | 3 |
| CTC | L | 0.351 | 26.000 | 13 |
| CTG | L | 0.162 | 12.000 | 6 |
| CTT | L | 0.108 | 8.000 | 4 |
| TTA | L | 0.027 | 2.000 | 1 |
| TTG | L | 0.270 | 20.000 | 10 |
| ATG | M | 1.000 | 22.000 | 11 |
| AAC | N | 0.885 | 46.000 | 23 |
| AAT | N | 0.115 | 6.000 | 3 |
| CCA | P | 0.136 | 6.000 | 3 |
| CCC | P | 0.364 | 16.000 | 8 |
| CCG | P | 0.227 | 10.000 | 5 |
| CCT | P | 0.273 | 12.000 | 6 |
| CAA | Q | 0.250 | 10.000 | 5 |
| CAG | Q | 0.750 | 30.000 | 15 |
| AGA | R | 0.000 | 0.000 | 0 |
| AGG | R | 0.300 | 6.000 | 3 |
| CGA | R | 0.200 | 4.000 | 2 |
| CGC | R | 0.200 | 4.000 | 2 |
| CGG | R | 0.200 | 4.000 | 2 |
| CGT | R | 0.100 | 2.000 | 1 |
| AGC | S | 0.162 | 12.000 | 6 |
| AGT | S | 0.108 | 8.000 | 4 |
| TCA | S | 0.108 | 8.000 | 4 |
| TCC | S | 0.243 | 18.000 | 9 |
| TCG | S | 0.270 | 20.000 | 10 |
| TCT | S | 0.108 | 8.000 | 4 |
| ACA | T | 0.250 | 20.000 | 10 |
| ACC | T | 0.325 | 26.000 | 13 |
| ACG | T | 0.200 | 16.000 | 8 |
| ACT | T | 0.225 | 18.000 | 9 |
| GTA | V | 0.129 | 8.000 | 4 |
| GTC | V | 0.387 | 24.000 | 12 |
| GTG | V | 0.323 | 20.000 | 10 |
| GTT | V | 0.161 | 10.000 | 5 |
| TGG | W | 1.000 | 24.000 | 12 |
| TAC | Y | 0.686 | 48.000 | 24 |
| TAT | Y | 0.314 | 22.000 | 11 |
| TAA | * | 0.000 | 0.000 | 0 |
| TAG | * | 0.000 | 0.000 | 0 |
| TGA | * | 1.000 | 2.000 | 1 |

### Introns

Eukaryotic genes may be interrupted by intervening sequences (introns) which must be modified in precursor transcripts in order to produce functional mRNAs. This process of intron removal is known as pre-mRNA splicing. Usually, a branchpoint sequence of an intron is necessary for intron splicing through the formation of a lariat. Signals for splicing reside directly at the boundaries of the intron splice sites. The boundaries of intron splice sites usually have the consensus intron sequences GT and AG at their 5′ and 3′ extremities, respectively. While no 3′ splice sites other than AG have been reported, there are reports of a few exceptions to the 5′ GT splice site. For example, there are precedents where CT or GC is substituted for GT at the 5′ boundary. There is also a strong preference for the nucleotide bases ANGT to follow GT where N is A, C, G, or T (primarily A or T in Saccharomyces species), but there is no marked preference for any particular nucleotides to precede the GT splice site. The 3′ splice site AG is primarily preceded by a pyrimidine nucleotide base (Py), i.e., C or T.

The number of introns that can interrupt a fungal gene ranges from one to twelve or more introns (Rymond and Rosbash, 1992, In, E. W. Jones, J. R. Pringle, and J. R. Broach, editors, The Molecular and Cellular Biology of the Yeast Saccharomyces, pages 143-192, Cold Spring Harbor Laboratory Press, Plainview, N.Y.; Gurr et al., 1987, In Kinghorn, J. R. (ed.), Gene Structure in Eukaryotic Microbes, pages 93-139, IRL Press, Oxford). They may be distributed throughout a gene or situated towards the 5′ or 3′ end of a gene. In Saccharomyces cerevisiae, introns are located primarily at the 5′ end of the gene. Introns may be generally less than 1 kb in size, and usually are less than 400 bp in size in yeast and less than 100 bp in filamentous fungi.

The Saccharomyces cerevisiae intron branchpoint sequence 5′-TACTAAC-3′ rarely appears exactly in filamentous fungal introns (Gurr et al., 1987, supra). Sequence stretches closely or loosely resembling TACTAAC are seen at equivalent points in filamentous fungal introns with a general consensus NRCTRAC where N is A, C, G, or T, and R is A or G. For example, the fourth position T is invariant in both the Neurospora crassa and Aspergillus nidulans putative consensus sequences. Furthermore, nucleotides G, A, and C predominate in over 80% of the positions 3, 6, and 7, respectively, although position 7 in Aspergillus nidulans is more flexible with only 65% C. However, positions 1, 2, 5, and 8 are much less strict in both Neurospora crassa and Aspergillus nidulans. Other filamentous fungi have similar branchpoint stretches at equivalent positions in their introns, but the sampling is too small to discern any definite trends.

The heterologous expression of a gene encoding a polypeptide in a fungal host strain may result in the host strain incorrectly recognizing a region within the coding sequence of the gene as an intervening sequence or intron. For example, it has been found that intron-containing genes of filamentous fungi are incorrectly spliced in Saccharomyces cerevisiae (Gurr et al., 1987, In Kinghom, J. R. (ed.), Gene Structure in Eukaryotic Microbes, pages 93-139, IRL Press, Oxford). Since the region is not recognized as an intron by the parent strain from which the gene was obtained, the intron is called a cryptic intron. This improper recognition of an intron, referred to herein as a cryptic intron, may lead to aberrant splicing of the precursor mRNA molecules resulting in no production of biologically active polypeptide or in the production of several populations of polypeptide products with varying biological activity.

“Cryptic intron” is defined herein as a region of a coding sequence that is incorrectly recognized as an intron which is excised from the primary mRNA transcript. A cryptic intron preferably has 10 to 1500 nucleotides, more preferably 20 to 1000 nucleotides, even more preferably 30 to 300 nucleotides, and most preferably 30 to 100 nucleotides.

The presence of cryptic introns can in particular be a problem when trying to express proteins in organisms which have a less strict requirement to what sequences are necessary in order to define an intron. Such “sloppy” recognition can result e.g. when trying to express recombinant proteins in fungal expression systems.

Cryptic introns can be identified by the use of Reverse Transcription Polymerase Chain Reaction (RT-PCR). In RT-PCR, mRNA is reverse transcribed into single stranded cDNA that can be PCR amplified to double stranded cDNA. PCR primers can then be designed to amplify parts of the single stranded or double stranded cDNA, and sequence analysis of the resulting PCR products compared to the sequence of the genomic DNA reveals the presence and exact location of cryptic introns (T. Kumazaki et al. (1999) J. Cell. Sci. 112, 1449-1453).

According to one embodiment of the invention the modification introduced into the wild type gene sequence will optimize the mRNA for expression in a particular host organism. In the present invention the host organism or host cell comprises a group of fungi referred to as filamentous fungi as explained in more detail below.

### Filamentous Fungal Host Cell

The host cell of the invention is a filamentous fungus represented by the following groups of Ascomycota, include, e.g., Neurospora, Eupenicillium (=Penicillium), Emericella (=Aspergillus), Eurotium (=Aspergillus).

In a preferred embodiment, the filamentous fungus includes all filamentous forms of the subdivision Eumycota and Oomycota (as defined by Hawksworth et al., In, Ainsworth and Bisby's Dictionary of The Fungi, 8th edition, 1995, CAB International, University Press, Cambridge, UK). The filamentous fungi are characterized by a vegetative mycelium composed of chitin, cellulose, glucan, chitosan, mannan, and other complex polysaccharides. Vegetative growth is by hyphal elongation and carbon catabolism is obligately aerobic.

In a more preferred embodiment, the filamentous fungal host cell is a cell of a species of, but not limited to, Acremonium, Aspergillus, Fusarium, Humicola, Mucor, Myceliophthora, Neurospora, Penicillium, Thielavia, Tolypocladium, and Trichoderma or a teleomorph or synonym thereof. In an even more preferred embodiment, the filamentous fungal host cell is an Aspergillus cell. In another even more preferred embodiment, the filamentous fungal host cell is an Acremonium cell. In another even more preferred embodiment, the filamentous fungal host cell is a Fusarium cell. In another even more preferred embodiment, the filamentous fungal host cell is a Humicola cell. In another even more preferred embodiment, the filamentous fungal host cell is a Mucor cell. In another even more preferred embodiment, the filamentous fungal host cell is a Myceliophthora cell. In another even more preferred embodiment, the filamentous fungal host cell is a Neurospora cell. In another even more preferred embodiment, the filamentous fungal host cell is a Penicillium cell. In another even more preferred embodiment, the filamentous fungal host cell is a Thielavia cell. In another even more preferred embodiment, the filamentous fungal host cell is a Tolypocladium cell. In another even more preferred embodiment, the filamentous fungal host cell is a Trichoderma cell. In a most preferred embodiment, the filamentous fungal host cell is an Aspergillus awamori, Aspergillus foetidus, Aspergillus japonicus, Aspergillus aculeatus, Aspergillus niger, Aspergillus nidulans or Aspergillus oryzae cell. In another preferred embodiment, the filamentous fungal host cell is a Fusarium cell of the section Discolor (also known as the section Fusarium). For example, the filamentous fungal parent cell may be a Fusarium bactridioides, Fusarium cerealis, Fusarium crookwellense, Fusarium culmorum, Fusarium graminearum, Fusarium graminum, Fusarium heterosporum, Fusarium negundi, Fusarium reticulatum, Fusarium roseum, Fusarium sambucinum, Fusarium sarcochroum, Fusarium sulphureum, or Fusarium trichothecioldes cell. In another preferred embodiment, the filamentous fungal parent cell is a Fusarium strain of the section Elegans, e.g., Fusarium oxysporum. In another most preferred embodiment, the filamentous fungal host cell is a Humicola insolens or Humicola lanuginosa cell. In another most preferred embodiment, the filamentous fungal host cell is a Mucor miehei cell. In another most preferred embodiment, the filamentous fungal host cell is a Myceliophthora thermophilum cell.

In another most preferred embodiment, the filamentous fungal host cell is a Neurospora crassa cell. In another most preferred embodiment, the filamentous fungal host cell is a Penicillium purpurogenum or Penicillium funiculosum (WO 00/68401) cell. In another most preferred embodiment, the filamentous fungal host cell is a Thielavia terrestris cell. In another most preferred embodiment, the Trichoderma cell is a Trichoderma harzianum, Trichoderma koningii, Trichoderma longibrachiatum, Trichoderma reesei or Trichoderma viride cell.

In a particular embodiment the filamentous host cell is an A. oryzae or A. niger cell.

In a preferred embodiment of the invention the host cell is a protease deficient or protease minus strain.

This may e.g. be the protease deficient strain Aspergillus oryzae JaL 125 having the alkaline protease gene named “alp” deleted. This strain is described in WO 97/35956 (Novozymes), or EP patent no. 429,490, or the TPAP free host cell, in particular a strain of A. niger, disclosed in WO 96/14404. Further, also host cell, especially A. niger or A. oryzae, with reduced production of the transcriptional activator (prtT) as described in WO 01/68864 is specifically contemplated according to the invention.

### Transformation of Fungi

Fungal cells may be transformed by a process involving protoplast formation, trans-formation of the protoplasts, and regeneration of the cell wall in a manner known per se. Suitable procedures for transformation of Aspergillus host cells are described in EP 238 023 and Yelton et al., 1984, Proceedings of the National Academy of Sciences USA 81: 1470-1474. Suitable methods for transforming Fusarium species are described by Malardier et al., 1989, Gene 78: 147-156 and WO 96/00787. Yeast may be transformed using the procedures described by Becker and Guarente, In Abelson, J. N. and Simon, M. I., editors, Guide to Yeast Genetics and Molecular Biology, Methods in Enzymology, Volume 194, pp 182-187, Academic Press, Inc., New York; Ito et al., 1983, Journal of Bacteriology 153: 163; and Hinnen et al., 1978, Proceedings of the National Academy of Sciences USA 75: 1920.

### Methods of Production

The present invention also relates to expression of the modified nucleic acid sequence in order to produce the serum albumin. Expression comprises (a) cultivating a filamentous fungus expressing the serum albumin from the modified nucleic acid sequence; and (b) recovering the polypeptide. Preferably, the filamentous fungus is of the genus Aspergillus, and more preferably Aspergillus oryzae or Aspergillus niger.

In the production methods of the present invention, the cells are cultivated in a nutrient medium suitable for production of the polypeptide using methods known in the art. For example, the cell may be cultivated by shake flask cultivation, small-scale or large-scale fermentation (including continuous, batch, fed-batch, or solid state fermentations) in laboratory or industrial fermentors performed in a suitable medium and under conditions allowing the polypeptide to be expressed and/or isolated. The cultivation takes place in a suitable nutrient medium comprising carbon and nitrogen sources and inorganic salts, using procedures known in the art. Suitable media are available from commercial suppliers or may be prepared according to published compositions (e.g., in catalogues of the American Type Culture Collection). If the polypeptide is secreted into the nutrient medium, the polypeptide can be recovered directly from the medium. If the polypeptide is not secreted, it can be recovered from cell lysates.

The polypeptides may be detected using methods known in the art that are specific for the polypeptides, such as N-terminal sequencing of the polypeptide. These detection methods may include use of specific antibodies. The resulting polypeptide may be recovered by methods known in the art. For example, the polypeptide may be recovered from the nutrient medium by conventional procedures including, but not limited to, centrifugation, filtration, extraction, spray-drying, evaporation, or precipitation.

The polypeptides of the present invention may be purified by a variety of procedures known in the art including, but not limited to, chromatography (e.g., ion exchange, affinity, hydrophobic, chromatofocusing, and size exclusion), electrophoretic procedures (e.g., preparative isoelectric focusing), differential solubility (e.g., ammonium sulfate precipitation), SDS-PAGE, or extraction (see, e.g., Protein Purification, J.-C. Janson and Lars Ryden, editors, VCH Publishers, New York, 1989).

In a further aspect the present invention relates to a modified nucleic acid sequence encoding the BSA protein and capable of expression in a filamentous fungal host organism, which modified nucleic acid sequence is obtainable by:

i) providing the wild type nucleic acid sequence encoding BSA;
ii) modifying at least one codon, wherein the modification does not change the amino acid encoded by said codon and the nucleic acid sequence of said codon is different compared to the corresponding codon in the wild type gene.

In an even further aspect the invention relates to a modified nucleic acid sequence encoding the HSA protein and capable of expression in a filamentous fungal host organism, which modified nucleic acid sequence is obtainable by:

i) providing the wild type nucleic acid sequence encoding HSA;
ii) modifying at least one codon, wherein the modification does not change the amino acid encoded by said codon and the nucleic acid sequence of said codon is different compared to the corresponding codon in the wild type gene.

Particularly the wild type sequences encoding BSA or HSA are the specific sequences shown in SEQ ID NO: 1 and 3.

In the present context the term “capable of expression in a filamentous host” means that the yield of the serum albumin protein should be at least 1.5 mg/l, more particularly at least 2.5 mg/l, more particularly at least 5 mg/A, more particularly at least 10 mg/l, even more particularly at least 20 mg/l, or more particularly 0.5 g/L, or more particularly 1 g/L, or more particularly 5 g/L, or more particularly 10 g/L, or more particularly 20 g/L.

Specific examples of modified nucleic acid sequences encoding serum albumin and modified according to the invention in order to provide expression of the serum albumin protein in a filamentous fungal host, like e.g. Aspergillus, are shown in SEQ ID NO: 5 (BSA) and 7 (HSA). The information disclosed herein will allow the skilled person to isolate other modified nucleic acid sequences following the directions above, which sequences can also be expressed in filamentous fungi and such sequences are also comprised within the scope of the present invention.

In one embodiment the present invention relates to a modified nucleic acid sequence according to the invention as shown in SEQ ID NO: 5.

In another embodiment the present invention relates to a modified nucleic acid sequence according to the invention as shown in SEQ ID NO: 7.

Variations in the choice of codon usage and the number of codons, which have been optimized can vary and still provide as nucleic acid sequence capable of expression in a filamentous fungi. Such alternative sequences will be homologous to the specific sequences shown in SEQ ID NO: 5 and 7.

In a further embodiment the invention therefore relates to a modified nucleic acid sequence encoding the wild type BSA protein and capable of expression in a filamentous fungal host organism, wherein:

a) the modified sequence has at least 77% identity with SEQ ID NO: 5; or
b) the modified sequence hybridizes under high stringency conditions with a polynucleotide probe consisting of the complementary strand of nucleotides 1 to 1821 of SEQ ID NO: 5.

In still another embodiment the invention relates to a modified nucleic acid sequence encoding the wild type HSA protein and capable of expression in a filamentous fungal host organism, wherein:

a) the modified sequence has at least 77% identity with SEQ ID NO: 7; or
b) the modified sequence hybridizes under high stringency conditions with a polynucleotide probe consisting of the complementary strand of nucleotides 1 to 1827 of SEQ ID NO: 7.

The modified nucleic acid sequence according to the invention has at 77% identity with the sequence shown in SEQ ID NO: 5, particularly at least 79% identity, more particularly at least 82%, more particularly at least 85%, more particularly at least 90%, more particularly at least 95%, more particularly at least 98%, even more particularly at least 99% identity.

The modified nucleic acid sequence according to the invention has at 77% identity with the sequence shown in SEQ ID NO: 7, particularly at least 79% identity, more particularly at least 82%, more particularly at least 85%, more particularly at least 90%, more particularly at least 95%, more particularly at least 98%, even more particularly at least 99% identity.

### Identity:

In the present context, the homology between two amino acid sequences or between two nucleic acid sequences is described by the parameter “identity”.

For purposes of the present invention, alignments of sequences and calculation of homology scores may be done using a full Smith-Waterman alignment, useful for both protein and DNA alignments. The default scoring matrices BLOSUM50 and the identity matrix are used for protein and DNA alignments respectively. The penalty for the first residue in a gap is −12 for proteins and −16 for DNA, while the penalty for additional residues in a gap is −2 for proteins and −4 for DNA. Alignment may be made with the FASTA package version v20u6 (W. R. Pearson and D. J. Lipman (1988), “Improved Tools for Biological Sequence Analysis”, PNAS 85:2444-2448, and W. R. Pearson (1990) “Rapid and Sensitive Sequence Comparison with FASTP and FASTA”, Methods in Enzymology, 183:63-98).

Multiple alignments of protein sequences may be made using “ClustalW” (Thompson, J. D., Higgins, D. G. and Gibson, T. J. (1994) CLUSTAL W: improving the sensitivity of progressive multiple sequence alignment through sequence weighting, positions-specific gap penalties and weight matrix choice. Nucleic Acids Research, 22:4673-4680). Multiple alignments of DNA sequences may be done using the protein alignment as a template, replacing the amino acids with the corresponding codon from the DNA sequence.

### Hybridization

For purposes of the present invention, hybridization indicates that the nucleic acid sequence hybridizes to a labeled polynucleotide probe which hybridizes to the nucleic acid sequence shown in SEQ ID NO: 5 or 7 under very low to very high stringency conditions. Molecules to which the polynucleotide probe hybridizes under these conditions may be detected using X-ray film or by any other method known in the art. Whenever the term “polynucleotide probe” is used in the present context, it is to be understood that such a probe contains at least 15 nucleotides.

In one embodiment, the polynucleotide probe is the complementary strand of SEQ ID NO: 5 or 7.

For long probes of at least 100 nucleotides in length, very low to very high stringency conditions are defined as pre-hybridization and hybridization at 42° C. in 5×SSPE, 1.0% SDS, 5×Denhardt's solution, 100 μg/ml sheared and denatured salmon sperm DNA, following standard Southern blotting procedures. Preferably, the long probes of at least 100 nucleotides do not contain more than 1000 nucleotides. For long probes of at least 100 nucleotides in length, the carrier material is finally washed three times each for 15 minutes using 2×SSC, 0.1% SDS at 42° C. (very low stringency), preferably washed three times each for 15 minutes using 0.5×SSC, 0.1% SDS at 42° C. (low stringency), more preferably washed three times each for 15 minutes using 0.2×SSC, 0.1% SDS at 42° C. (medium stringency), even more preferably washed three times each for 15 minutes using 0.2×SSC, 0.1% SDS at 55° C. (medium-high stringency), most preferably washed three times each for 15 minutes using 0.1×SSC, 0.1% SDS at 60° C. (high stringency), in particular washed three times each for 15 minutes using 0.1×SSC, 0.1% SDS at 68° C. (very high stringency).

Although not particularly preferred, it is contemplated that shorter probes, e.g. probes which are from about 15 to 99 nucleotides in length, such as from about 15 to about 70 nucleotides in length, may also be used. For such short probes, stringency conditions are defined as prehybridization, hybridization, and washing post-hybridization at 5° C. to 10° C. below the calculated Tₘ using the calculation according to Bolton and McCarthy (1962, Proceedings of the National Academy of Sciences USA 48:1390) in 0.9 M NaCl, 0.09 M Tris-HCl pH 7.6, 6 mM EDTA, 0.5% NP-40, 1×Denhardt's solution, 1 mM sodium pyrophosphate, 1 mM sodium monobasic phosphate, 0.1 mM ATP, and 0.2 mg of yeast RNA per ml following standard Southern blotting procedures.

For short probes which are about 15 nucleotides to 99 nucleotides in length, the carrier material is washed once in 6×SCC plus 0.1% SDS for 15 minutes and twice each for 15 minutes using 6×SSC at 5° C. to 10° C. below the calculated Tₘ.

### Albumin as a Dried Product

The present invention relates in a further embodiment to a dried and fast dissolving particle comprising serum albumin, hereinafter referred to as SA, as well as to various compositions and articles comprising the dry fast dissolving particle or said compositions of this embodiment. The present invention further relates to methods for the preparation of fast dissolving SA particle of the invention, and uses thereof.

The SA can be any form of serum albumin like e.g. human serum albumin or bovine serum albumin, and the SA species can be natural or recombinant serum albumin.

We have surprisingly found that by spray drying we obtain a product wherein the SA does not change its properties during the process step. However during simple spray drying primary particles with a very low particle size are obtained. Said primary particles can not readily disperse in liquids. The primary particles stick together and make a gel like substance when added to a liquid. To get the primary particles dispersed mechanical stirring over a prolonged period of time is required. Thus it would be desirable to obtain a SA product which readily disperses when added to a liquid and is thus fast dissolving.

In our search to find a dried powder of SA which dissolved instantly in aqueous solutions, we have surprisingly found that by enlarging the SA particles, e.g. by agglomerating primary particles of SA, we obtain a product which is readily soluble in aqueous solutions.

A “primary particle” of SA is a particle which has particle size less than 10 microns and e.g. has been prepared by spray drying one droplet of liquid.

An “agglomerate” is defined as a particle comprising at least two primary particles bonded together.

The “dispersibility” is measured in the following way: A dry HSA product corresponding to 1.0 g of pure HSA (or other serum albumin) is added to 100 ml of water (25° C.) in a 600 ml beaker during gentle manual stirring with a spoon. The time for complete dissolution is measured in seconds and used as the dispersibility.

The “solubility” is measured in the following way: A dry HSA (or other serum albumin) product corresponding to 2.5 g of pure HSA product is added to 100 ml water (25° C.) and gently stirred with a spoon for 20 sec. A sample of the liquid phase is taken and the dry matter content is measured. The percentage of solids in the solution is calculated as percentage of the total added solids. A solubility of 100% corresponds to all added solids being dissolved in less than 20 sec.

The “wettability” is measured in the following way: A dry HSA (or other serum albumin) product corresponding to 0.5 g of pure HSA product is added to the surface of 100 ml water (22° C.) in a 600 ml beaker. The time for complete dissolution is measured in seconds and the value is the wettability.

“Bulk density” is the mass of powder per volume measured in a cylindrical container as specified in the norm: DIN 52102.

“Tapped density” is the mass of powder per volume measured in cylindrical container which is tapped a number of times as defined in the norm: ISO 787/11.

The present invention shows that a controlled agglomeration process of a dried SA product greatly improves the solubility of the product, thus achieving very fast dissolution in a solvent, which preferably is water.

### The Dried SA Product

The SA product of the present invention has preferably a particle size between 10 and 1,000 microns. In a particular embodiment the particle size is between 50 to 500 microns. In a more particular embodiment the particle size is between 100 and 250 microns as determined by laser diffraction measurement of the particles suspended in isopropanol, as shown in the examples below. In a particular embodiment the particle size is above 50 microns. In a more particular embodiment the particle size is above 100 microns. In a particular embodiment the particle size is below 700 microns.

In a particular embodiment of the present invention the dried product has a particle size, 50 percentile, D₅₀, which is between 10 and 1,000 microns, preferably between 100 and 1,000 microns, more preferably between 150 and 900 microns, and even more preferably between 200 and 800 microns, as determined by laser diffraction measurement of the particles suspended in isopropanol, as shown in the examples below.

It is desirable to have a narrow span of the particles as too large particles take too long time to dissolve and too small particles stick together and are also very difficult to dissolve.

In a particular embodiment, the polydispersity of the spray dried and agglomerated product is measured as the SPAN value, which is calculated according to the following formula: SPAN=(D₉₀−D₁₀)/D₅₀. In a particular embodiment of the present invention the SPAN value is less than 2.5. In a more particular embodiment the SPAN value is less than 2.0. In an even more particular embodiment the SPAN value is less than 1.5. In a most particular embodiment the SPAN value is less than 1.2.

In the above D₁₀ is the average particle size where 10% of the mass of particles are smaller than or equal to this size. D₅₀ is the average particle size where 50% of the mass of particles are smaller than or equal to this size. D₉₀ is the average particle size where 90% of the mass of particles are smaller than or equal to this size.

The amount of SA present in the dried SA agglomerate of the present invention is in a particular embodiment more than 50%. In a more particular embodiment of the present invention the SA present in the dried SA agglomerate is more than 75%. In a more particular embodiment of the present invention the SA present in the dried SA agglomerate is more than 90%. In a most particular embodiment of the present invention the SA present in the dried SA agglomerate is more than 95% even more than 99. In a particular embodiment the SA present in the dried SA agglomerate is 100%.

In a particular embodiment the particles dissolve within 5 minutes. In a more particular embodiment the particles dissolve within 3 minutes. In an even more particular embodiment particles dissolve within 1% minutes. In a most particular embodiment the particles dissolve within ½ minutes.

The dried SA agglomerate of the present invention may also comprise other ingredients. Other ingredients may be but are not limited to fillers, agglomeration aids, one or more active ingredients, such as pharmacologically active substances, and water-soluble excipients, buffer, salts, surfactants, polymers e.g. polyethylene glycol, carbohydrates e.g. starch, mannitol, sorbitol, inorganic salt such as sodium chloride, phosphate and a mixture thereof. Nutrients for cells, such as salts and sugars for cell culture media, said other ingredient may be added to the liquid composition comprising the SA.

Further ingredients, which may be added to the liquid composition is heat stabilizers for SA e.g. octanoate or N-acetyl-tryptophan.

For all uses, the albumin may be spray-dried with a combination of one or more compounds, including but not limited to pharmaceutical actives, e.g. to antigens, antibodies, antibody fragments, hormones, growth factors, interleukins Betaferon (interferon-1b), human erythropoietin (EPO), glucocerebrosidase, insulin, in particular recombinant insulin produced in E. coli and S. cerevisiae, Urate oxidase, Glucagon, Granulocyte-macrophage colony stimulating factor, Hirudin, lepirudin, Platelet-derived growth factor, Antiangiogenic factor, Antibodies (mAb and/or PAb), antibody fragments (Fab), Anti microbial peptides, anti viral peptides, anti-cancer peptides, IGF (insulin like growth factor e.g. IGF-1, IGf-2), EGF (epidermal growth factor), TGF (TGF-alfa) Transforming growth factor, Heparin, Interleukins, Cytokines, Hormones (including but not limited to sex hormones, such as Follicle Stimulating hormone and Human chorionic gonadotrophin, human growth hormone, peptide hormones, cortison hormones), Antigens (e.g. HIV, HBV, HCV, HPV, Dengue Virus, Malaria, Hepatitis B surface antigen, Measles, Rubella, Mumps), Botulinum toxins, Elastin, Gellatine, Collagen, Protein A, Lactic acid, Hyaloronic acid, human intrinsic factor.

### The Liquid Composition

The liquid composition to be formulated into a dry, fast dissolving product comprises SA. The liquid composition may further comprise other ingredients.

The dry solid content of the liquid composition may vary between 1 and 30 wt %. In a particular embodiment of the present invention the dry solid content of the liquid composition varies between 3 and 15 wt %. If the dry solid content is too high the liquid composition becomes too viscous and it will be difficult to handle.

The liquid composition may comprise other ingredients. In a particular embodiment of the present invention the amount of other ingredients does not exceed 50 wt % of the dry solids. In a more particular embodiment of the present invention the amount of other ingredients does not exceed 25 wt %.

In a particular embodiment the liquid composition is aqueous.

### Method of Producing Dried SA Particles

Normally, when spray drying particles the particles obtained are primary particles and they are very small e.g. below 10 microns. Said primary particles are very difficult to disperse and dissolve in aqueous liquid. However we have found that larger particles disperse and dissolve readily in aqueous water. We have found that it is possible to prepare enlarged particles e.g. by agglomeration of primary particles or by running the spray drying process under specific conditions whereby the droplets prepared by atomization are very big and thereby the dried particles obtained also are bigger compared to particles obtained by spray drying run under normal conditions. By using the latter process it is not necessary to include a fluid bed.

The present invention provides a method for producing a dry and fast dissolving SA product and/or preferred embodiments thereof.

In one embodiment of the present invention the method comprises the steps of:

a) drying a liquid composition comprising serum albumin; and
b) agglomerating the product of a).

In another embodiment of the present invention the method comprises the steps of
a) preparation of a liquid composition comprising serum albumin;
b) atomization of the liquid composition of step a)
c) drying of the liquid composition to obtain particles with a particle size above 50 microns.

In a particular embodiment of the present invention the atomization in step b) is performed by use of a two fluid nozzle atomizer, a pressure nozzle or a rotary atomizer.

To obtain particles with a particle size above 50 microns by use of a two fluid nozzle atomizer or a pressure nozzle the atomization pressure has to be low. In a particular embodiment of the present invention the atomization pressure is below 5 bar in a more particular embodiment of the present invention the atomization pressure is below 3 bar.

To obtain particles with a particle size above 50 microns by use of a rotary atomizer the rotation speed has to be low. In a particular embodiment the rotation speed has to be below 100 m/s. In a more particular embodiment the rotation speed has to be below 50 m/s.

Drying of the SA containing liquid composition may be achieved by any drying method available to a skilled person such as spray drying, freeze drying, vacuum drying, fluid bed drying and microwave drying. It is preferred that a fast drying method is used. If a slow drying method is used the risk of starting protein polymerization is high.

In a particular embodiment spray drying is used in the drying step as spray drying is a fast process and it is possible to add gas into the liquid formulation to vary the porosity of the obtained particles.

The drying and enlargement of the particles may take place in, but are not limited to, process equipment like spray dryers, fluid beds and or equipment including both processes wherein both are combined such as fluid bed spray dryers (fluidized spray dryer).

In a particular embodiment of the present invention a fluidization of the particles is included in the process.

In a particular embodiment the present process combines the principles of drying processes and agglomeration processes. In a particular embodiment a spray dryer and a fluid bed is used.

In a particular embodiment of the present invention these two principles are combined into one piece of equipment e.g. a fluidized spray dryer. To save process time it is convenient that the drying and agglomerating is done in one step. In a particular embodiment of the present invention the drying and agglomeration is done in a one process step by using a fluidized spray dryer. Typically the inlet temperature is between 50-200° C. The air outlet temperature is 20-90° C.

In a particular embodiment of the present invention the particles e.g. primary particles, to be re-cycled are blown into the atomization zone in the spray dryer wherein the agglomeration occurs.

In another particular embodiment the fluid bed is equipped with nozzles which are spraying the liquid composition onto the primary fluidized particles formed in the fluid bed whereby agglomerates are formed.

In a particular embodiment of the present invention the invention discloses a process for the production of agglomerated SA wherein a liquid composition is prepared comprising SA, optionally additives are added to the composition. One or more of said liquid compositions are sprayed into a fluidized bed. In a particular embodiment the liquid compositions are sprayed into a fluidized bed from below by means of spray nozzles. Fine material that escapes from the fluid bed with the off gas is separated and returned to the fluidised bed as nuclei for the agglomerates. Agglomerates of a pre-determined size are formed by adjusting the sifting gas stream and the finished agglomerates are discharged.

It may be convenient first to produce the dry fine powder by e.g. spray drying. Said fine powder is fluidized in a fluid bed and a liquid binder e.g. water is sprayed into the equipment to build up the desired agglomerates.

After drying the obtained fast dissolving SA particles/agglomerate is removed from the process.

In a particular embodiment of the present invention it is necessary to include a re-cycling system. Said recycling system may recycle primary particles or particles which in general are too small or particles which have been grinded as they are too big. The system works preferably by classifying the dried particles in two or more size classes wherein the particles which are too small are re-cycled and the particles of the desired size are removed from the process. The recycling system may work as an internal system and/or an external system.

In an external system the material to be re-cycled escaping from the fluidised bed may be continuously separated off from the off air with the aid of a cyclone separator or dust filter and returned to the fluidised bed. In an internal system the material to be re-cycled is effected with aid of a dust filter arranged above the fluidised bed.

The process may include the grinding of oversized particles and return of the grinded particles to the fluidized bed by an external system.

The recycling system may work as a wet or dry recycling system. In a wet re-cycling system the re-cycled particles are dissolved or suspended into the in-going liquid composition. In a dry re-cycling system the particles are returned back into the atomization zone.

In a particular embodiment the technique used is a spray dryer equipped with a Walzel type of atomizer and a re-cycle system.

It has been found that by introducing a gas into the particles during spray drying it is possible to vary the porosity of the obtained particles. Porous particles are normally faster dissolving than non-porous particles. It is thus possible to vary the solubility of the spray dried particles. The process may include a process step where a gas is introduced into the liquid composition. In a particular embodiment the gas to be introduced is carbon dioxide, nitrogen, or atmospheric air.

### Serum Albumin Applications

Albumin, whether obtained recombinantly or from plasma sources, can be used for a number of applications. It is known to exhibit a variety of functions both in vitro and in vivo. (Kragh-Hansen U, Chuang V T, Otagiri M (2002). Practical aspects of ligand-binding and enzymatic properties of human serum albumin. Biological & Pharmaceutical Bulletin. 25(6): 695-704; Curry S (2002). Beyond expansion: structural studies on the transport roles of human serum albumin. Vox Sanguinis. 83:Suppl-9; Nicholson J P, Wolmarans M R, Park G R (2000). Brit J Anaesth 85(4): 599-610; Peters, Theodore. All about albumin: biochemistry, genetics, and medical. Academic Press 1996, ISBN 0-12-552110-3)

In some applications, particularly in applications where there is a desire to use non-animal origin components, it is advantageous to combine recombinantly produced albumin with some or all of the following compounds when they are produced recombinantly or naturally by microbial systems: insulin, transferrin, IGF1, EGF or other proteins, growth factors and metabolites.

The spray-dried SA particles/agglomerate or/and the recombinantly produced SA according to the invention will be useful either as a final product or for production of albumin containing products used for:
Cell culture media
component in diagnostic kits
stabilizer of protein solutions
applications within the pharmaceutical area such as blood expanders and excipients
digestive support
removal of toxins
imaging—radiologic or ultrasonic imaging
drug delivery
coating of surfaces e.g. medical devices
invitro fertilization—both as storage medium of egg alone, sperma alone, but also for culturing of egg+sperma.

The serum albumin according to the invention can replace albumin derived from any animal species, most particular from human or bovine sources, or recombinant animal albumins, at an equivalent or better function for all uses of albumin. The reasons for this includes: 1) The invention, as it is herein described, naturally creates beneficial species of small molecules bound to the albumin molecules, including, but not limited to, molecules such as fatty acids, vitamins, amino acids, phospholipids and cations; and 2) It does not contain the high amounts of caprylic acid and N-acetyl DL tryptophan that many manufacturers of native and recombinant albumin use as stabilizers. It is well known that fatty acid and cation binding to albumin produce conformational changes which further affect both cooperative and competitive interactions of fatty acids and drugs. Excessive amounts of caprylic acid and/or N-acetyl DL tryptophan often have unwanted or adverse effects in many albumin applications. The use of recombinant human albumin in critically ill patients has thus been shown to increase mortality. (Olsen H, Andersen A, Nordbo A, Kongsgaard U E, Bormer O P, 2004. Pharmaceutical grade albumin: impaired drug binding capacity in vitro. BMC Clinical Pharmacology, 4:4 doi:10:1186/1472-69044-4; Keenan J, Dooley M, Pearson D, Clynes M. Recombinant human albumin in cell culture: Evaluation of growth promoting potential for NRK and Scc-9 cells in vitro. Cytotechnology 1997, 24:243-52; Zunszain, P A, Monie T, Konarev P V, Svergun D V, Curry S (2003). Structural analysis of conformational changes in human serum albumin associated with ligand binding and pH. www-hasylab.desy.de?science/annual_report/2003_report/part2/contrib./73/9952.pdf).

### Applications for SA

The applications for SA according to the present invention include but are not limited to:

i) the culture of mammalian cells for research, diagnostic or therapeutic purposes; the culture of genetically engineered or non-genetically engineered mammalian cells, including but not limited to CHO, Sp2/0, NS0, BHK, HEK 293, Namalwa and PERC.6, A431, for the production of biopharmaceuticals, diagnostic reagents or native or recombinant proteins, or adenoviruses to be used for medical or cosmetic purposes, and culture media for the same; the culture of normal primary human cells, for example those offered commercially from Clonetics, Cascade or Cell Applications, and culture media for the same; the culture of stem cells, for example cells available from Stem Cell Technologies or patient derived samples for bone marrow trans-plants or myocardial infarct repair, and culture media for the same; the culture of mammalian fibroblasts with and without kerotinocytes, for example Dermagraft® from Smith+Nephew, and the culture media for the same; the culture and expansion of mammalian tissue for implant and lesion repair, for example autologous chondrocyte implantation or myocyte implantation, and culture media for the same; (Yamane I. 1978. Development and application of a serum-free culture medium for primary culture. In H. Katsuta (ed), Nutritional Requirements of Cultured Cells. Baltimore, University Park Press, pp 1-21; U.S. Pat. No. 5,021,349; Iscove N N, Melchers F (1978). Complete replacement of serum by albumin, transferrin and soybean lipid in cultures of lipopolysaccharide-reactive B lymphocytes. J Exp Med 147: 928-33; U.S. Pat. No. 5,198,349; U.S. Pat. No. 5,250,421; Berntorp E (1997). Thrombosis Haemostasis 78: 256-60; McGrew J T, Richards C L, Smidt P, Dell B and Price V. 1998. Lipid requirements of a recombinant Chinese Hamster Ovary Cell Line (CHO), ibidem, pp 205-207; Yamane I. 1978. Role of bovine albumin in a serum-free culture medium and its application. Natl Cancer Inst Monogr 48:131-133; Sato J D, Kawamoto T, McClure D B and Sato G H. 1984. Cholesterol requirement of NS-1 mouse myeloma cells for growth in serum-free medium. Mol Biol Med 2(2):121-134; Kovar J. Hybridoma cultivation in defined serum-free media: growth-supporting substances. IV. Lipids and serum albumin. Folia Biol (Praha). 1987, 33(6):377-84; Jaeger, V, Lehmann J, Friedl P. Serum-free growth medium for the cultivation of a wide spectrum of mammalian cells in stirred bioreactors. Cytotechnology 1988, 1:319-29; Glassy C M, Tharakan J P, Chau, P C. Serum-free media in hybridoma culture and monoclonal antibody production. Biotech Bioeng 1988, 32:1015-28).
ii) use as a cryoprotectant for mammalian cells; (Somlo G, et al (1997). Effect of CD34+ selection and various schedules of stem cell reinfusion and granulocyte colony stimulating factor priming on hematopoetic recovery after high-dose chemotherapy for breast cancer. Blood 89: 1521-8; U.S. Pat. No. 6,548,297; WO01/37655; JRH Biosciences Catalog, 2004. Section on general cell culture techniques.
iii) use in or for process solutions used in mammalian assisted reproduction techniques; (Armstrong J S, Rajasekaran M, Hellstom W J G, Sikka S C (1998). Antioxidant potential of human serum albumin: role in recovery of high quality spermatozoa for assisted reproductive technology. J Androl 19:412-9; VandeVoort C A (2004). High quality sperm for non-human primate ART: Production and assessment. Reproduct Biol Endocrinol, 2: 33-8; Lane M, Maybach J M, Hooper K, Hasler J F, Gardner D K (2002). Cryo-survival and development of bovine blastocysts are enhanced by culture with recombinant albumin and hyaluronan Molecular Reproduction & Development 64: 70-8; Gardner, D K (2004). U.S. Pat. No. 6,762,053. Mammalian gamete and embryo culture media and culture media supplements.
iv) use in solutions for the preservation of donor organs; (US patent application 20040029096).
v) use in ocular applications; (Shimmura S, Ueno R, Matsumoto Y, Goto E, Higuchi A, Shimazaki J, Tsubota K (2003). Albumin as a tear supplement in the treatment of severe dry eye. Brit J Opthalmology 87:1279-83; U.S. Pat. No. 6,043,213).
vi) use in therapeutic applications as a plasma expander or for osmotic control, similar to the products Buminate (Baxter Intl), Plasbumin (Bayer Corp.) and Hextend (BioTime); (Woodruff L M, Gibson S T (1942). The clinical evaluation of human albumin. US Navy Med Bull 40:791-6; Heyl J T, Gibson J G II, Janeway C A (1943). Studies on the plasma proteins. V. The effect of concentrated solutions of human and bovine serum albumin on blood volume after acute blood loss in man. J Clin Invest 22: 763-73; Alderson P, Bunn F, Lefebvre C, Li Wn Po A, Li L, Roberts I, Schierhout G (2004). Human albumin solutions for resuscitation and volume expansion in critically ill patients. The Cochrane Database of Systematic Reviews, Issue 4. Art No. CD011208.pub2.DOI: 10.1002/14651858.CD001208.pub2)
vii) use as an excipient in the manufacture or formulation of pharmaceuticals or as a carrier, protecting agent, stabilizer or other use involving non-covalent association of another molecule, such as a drug, peptide or protein with the albumin (for example albumin-bound paclitxel suspension), for diagnostic or therapeutic use, including hormones (for example IGF-1 or insulin) and cytokines; (Tarelli E, et al (1998). Recombinant human albumin as a stabilizer for biological materials and for the preparation of international reference reagents. Biologicals 26: 33146; Paul W, Sharma C P (2005). Bioceramics, Towards Nano-enabled Drug Delivery: A mini Review. Trends Biomater. Artif Organs, 19: 7-11; Roddie, P H, Ludlam C A (1997). Blood Reviews 11:169-77).
viii) use in cosmetics or medical cosmetic procedures; (Sidle D M, Loos B M, Ramirez A L, Kabaker S S, Maas C S (2005). Use of BioGlue Surgical Adhesive for brow fixation in endoscope browplasty. Arch Facial Plast Surg 7: 393-7).
ix) use for inclusion in, on, or in the manufacturing of medical devices, including dental or dental implant applications, bone repair materials and biocompatible substances; (Kinnari T J, Rihkanen H, Laine T, Salonen, E-M, Jero, J (2004). Albumin-coated tympanostomy tubes: Prospective, double-blind clinical study. Laryngoscope 114: 2038-43; US patent application 20030004105.
x) use as a reagent in diagnostic procedures, kits or methods, for the purposes including but not limited to blocking non-specific adsorption of substances to surfaces, for local pH and osmolarity control in solution, to increase temperature stability of diagnostic or assay reagents, as a non-specific enzyme or small molecule stabilizer, as a stabilizer in the freezing or freeze-drying of small molecule, peptide and protein reagents.
xi) in the manufacture of human or veterinary vaccines, for example in Merck's MMR-II and MUMPSVAX vaccines, rabies vaccines, hepatitis A vaccine, the immunostabilization of virus in polymerized albumin; (U.S. Pat. No. 6,884,422; The BSE Inquirey: The Report (2000). Volume 16 chapter 4. www.bseinquirey.gov.uk).
xii) use as a standard or reference material; (Bradford M (1976). A rapid and sensitive method for the quantitation of microgram quantities of protein utilizing the principle of dye-binding. Anal Biochem 72: 248-54).
xiii) use in or for adhesives or sealants, similar to CryoLife BioGlue C or albumin fixatives; (Fuerst W, Banedjee A (2005). Release of glutaraldehyde from an albumin-glutaraldehyde tissue adhesive causes significant in vitro and in vivo toxicity. Ann Thorac Surg 79:1522-8; Passage J, Tam R, Windsor M, O'Brien M (2005). BioGlue: A review of the use of this new surgical adhesive in thoractic surgery. ANZ J Surg 75:315-8; Hoffman G T, et al (2004). Composites containing albumin protein or cyanoacrylate adhesives and biodegradable scaffolds: I. Acute wound closure study in a rat model. Proc SPIE, 5312:117-23); Medical Adhesives & Sealants (2003). Study #1681. The Freedonia Group).
xiv) use in the removal of toxins (Cole and Lirenman, 1978, J. Pediatr. 92:955-957)
xv) all other human or non-human applications and uses where an extracted or a recombinant mammalian albumin could be employed;

### Loading of Serum Albumin

However, in some applications, particularly applications where albumin is used as a cell culture ingredient, it is advantageous to load the albumin molecule with one or more ligands, including but not limited to fatty acids, vitamins, hormones and ions—e.g. copper, zink etc.

In some applications, it is advantageous to process albumin. Examples of such processing are:
Chromatographic purification, such as cat- and anion-exchange chromatography, affinity chromatography, reverse phase chromatography and mixed mode chromatography. The chromatography mode may, e.g., be packed bed or expanded bed adsorption/affinity chromatography.
Treatment with activated carbon and dextran coated charcoal or solvent extraction
Heating or pasteurisation in the presence of a stabilizer, e.g. caprylic acid and/or N-acetyltryptophan
Ultrafiltration
Protection of the protein from proteolytic degradation by addition of protease inhibitors.

Using these and other techniques, it may be possible to:

Reduce the colour of albumin, strip off non-covalently bound ligands, remove unwanted impurities, e.g. nucleic acids, enzyme activities—such as protease activities—carbohydrates, endotoxins and host cell protein as well as albumin derived impurities. The invention can be modified further by depleting the molecules that are not covalently bound to it and then reconstituting the albumin bound molecules specifically for the intended application to ensure a more optimum function. For cell cultures applications, albumin which is depleted of small molecules that remain bound from the manufacturing process has been shown to perform better for a given application when it is reconstituted with physiologically relevant mixtures of free fatty acids, phospholipids, cholesterols, hormones, metal ions, vitamins or drugs, more specifically one or more of linoleic acid, oleic acid, cholesterols, folic acid, cobalamin, pyridoxine, thyroxine, calcium (II)copper (II) and zinc (II).

(Rumsey S C, Galeano N F, Lipschitz B, Deckelbaum R J. (1995) J Biol Chem 270:10008-16; Bhattacharya M, Curry S, Franks N P. Binding of the general anesthetics propofol and halothane to human serum albumin: high resolution crystal structures. J Biol Chem 2000, 275:38731-8; Bhattacharya A A, Gruene T, Curry S. Crystallographic analysis reveals common modes of binding medium and long-chain fatty acids to human serum albumin. J Mol Bio 2000, 303:721-32. Synopsis: Similar to the method in C.2.2; Petitpas I, Gruene T, Bhattacharya A A, Curry, S. Crystal structures of human serum albumin complexed with monounsaturated and polyunsaturated fatty acids. J Mol Bio 2001, 314:955-60; Li Z, Zhuang J, Corson D W. Delivery of 9-Cis retinal to photoreceptors from bovine serum albumin. Photochem Photobiol. 1999, 69(4):5004. Albumin is also known to carry T4, folate and vitamin B12. Albumin-related gene products are known to carry vitamin D and vitamin K; Viscardi R M, Ullsperger S, McKenna M C. Carbon stripping extracts serum free fatty acids: implications for media supplementation of cultured type II pneumocytes. Lab Invest. 1991, 65(2):250; Caro J F, Hodges J, Sinha M K. Increased insulin responsiveness in isolated rat hepatocytes incubated with free fatty acid-poor albumin. Horm Metab Res. 1991, 23(8):362-4).

Moreover, in some applications, it is advantageous to enrich for certain covalent modifications of albumin—either by purification, chemically or biological strategies. Such modifications include reacting mercaptalbumin with cysteine or glutathione and using a host that predominantly produces a desired species rSA. Albumin blocked by cysteine or glutathione have improved heat stability, which is advantageous in certain applications or processing steps, e.g. in connection with heat denaturation of unwanted proteins—particularly unwanted enzymes.

Surprisingly, rSA produced by Aspergillus oryzae by recombinant expression, was found to support growth of mammalian cells in in vitro cultures—without prior loading—to an extent that exceeds that of unloaded as well as naturally loaded nSA. Even more surprisingly, HSA incubated with extracts from Aspergillus oryzae resulted in albumin that supports proliferation to an even greater extent. It is generally accepted that loading albumin with fatty acids confers a growth and often also productivity enhancing effect to mammalian cell cultures. rHSA from Aspergillus oryzae was surprisingly found to be loaded with linoleic acid, which is an essential fatty acid that in many applications will provide a positive contribution to growth and productivity of cell cultures. Some or all of these performance enhancing properties of rHSA from filamentous fungi can be further amplified by incubating albumin preparations, from any source, with extracts from filamentous fungi.

Serum albumin contacted with extracts derived from cultures of filamentous fungi will exhibit a growth- and probably also productivity-enhancing effect when added to cell culture medium.

Serum albumin loaded as described above is modified in a way that is particularly useful for application in cell culture medium, more particularly in serum free cell culture medium for primary cultures.

“Loading” in the context of the present invention means any modification to the mature serum albumin polypeptide, which includes addition of fatty acids but could also include addition of other factors, which modification takes place either when the serum albumin is expressed in a filamentous fungi, particularly an Aspergillus sp, or when any serum albumin, native, recombinant or fatty acid free, is contacted with a lysate derived from a culture of a filamentous fungi.

In one aspect the invention therefore relates to a loaded serum albumin obtainable by:

i) recombinant expression of a nucleic acid sequence encoding the serum albumin in a filamentous fungal host cell; and/or
ii) loading the serum albumin by contacting said serum albumin with a cell extract derived from filamentous fungal cells.

The loading can either be achieved by the recombinant expression of serum albumin in a suitable host cell or by contacting serum albumin from any source with a cell extract derived from a filamentous fungus.

The cell extract can in one embodiment be obtained by lysing the filamentous fungal host cells.

It is obvious to the skilled person that it is most efficient to use a cell lysate whereby the cells are actively disrupted, however, due to secretion and spontaneous lysis of cells in culture it is very likely that the culture medium as such could also be applied for loading serum albumin.

In a particular embodiment loading is achieved by contacting the serum albumin with culture broth used for culturing a filamentous fungus.

The term “cell extract” in the present context thus comprises culture broth from a cell culture of a filamentous fungi as well as a lysate obtained from a cell culture of a filamentous fungi or even a combination of both.

The filamentous fungal host cell is in one embodiment selected from the group consisting of Acremonium, Aspergillus, Fusarium, Humicola, Mucor, Myceliophthora, Neurospora, Penicillium, Thielavia, Tolypocladium, or Trichoderma.

Particularly the Aspergillus cell is Aspergillus awamori, Aspergillus foetidus, Aspergillus japonicus, Aspergillus niger, Aspergillus nidulans, or Aspergillus oryzae.

In one particular embodiment the loaded serum albumin is HSA or BSA.

In another particular embodiment the HSA or BSA is recombinantly produced in the filamentous fungi from the nucleic acid sequences selected from the group consisting of SEQ ID NO 5 and SEQ ID NO 7.

Loaded serum albumin according to the invention has been shown to be very useful when used in cell culture medium, thereby improving parameters such as cell viability, cell growth, and/or productivity.

The amount of SA added to cell culture medium is usually in the range from 0.1-100.000 μg/ml, particularly from 1-10.000 μg/ml, more particularly from 100-10.000 μg/ml medium.

In one embodiment the invention relates to a use of loaded serum albumin according to the invention in a cell culture medium. Particularly in serum free cell culture medium.

### Materials and Methods

### Materials

### Strains

MBin115 is described in WO 2004/090155, example 9.

### Example 1

### Cloning of the Gene Encoding Human Serum Albumin (HSA)

HSA has many applications such as media component or for drug delivery.

It is of interest to see if HSA can be expressed at economically interesting levels by the currently used hosts, especially the filamentous fungi.

Human adult male liver first strand cDNA was ordered and received from Stratagene (cat. no. 780621). The following two DNA oligo's where ordered and received:

190203J1

SEQ ID NO: 9:

ATGGACGGATCCACAATGAAGTGGGTAACCTTTATTTCC
190203J2

SEQ ID NO: 10:

ATGGACCCGCGGCTCGAGTTATAAGCCTAAGGCAGCTTGACTTGC

The following PCR was run using the two DNA oligoes and the cDNA as template along with the Pwo-polymerase (Roche); 94° C. 5 min 25* (94° C. 30 sec, 55° C. 30 sec, 72° C. 3 min) 72° C. 7 min.

The resulting PCR fragment was cloned into pCR4 blunt using the TOPO kit as recommended by manufacture (Invitrogen, cat no. 601059) resulting in plasmid pEN13046.

The gene was sequenced (see sequence at the end)

The cloned HSA has no N-glycosylation site.

The plasmid pEN13046 was cut with BamHI and SacI and the gene was isolated from agarose gel. The plasmid pEN12516 (WO 2004/069872) was cut BamHI and SacII and isolated from agarose gel. The vector and the gene was ligated and transformed into DH10b. The resulting plasmid was named pEN13054.

### Example 2

### Transformation of HSA cDNA Expression Plasmid into Aspergillus oryzae

The plasmid pENI3054 was transformed in to the Aspergillus oryzae strain JA1355 (WO 2004/069872). This was done as mentioned in WO 2004/069872.

10 transformants were grown in 200 μl YPM in a 96 well microtiter dish for 3 days at 34° C.

20 μl of supernatant was run on SDS-PAGE to see if the HSA was expressed by A. oryzae. No bands were detectable.

### Example 3

### Transformation of HSA cDNA Expression Plasmid into Aspergillus niger

The plasmid pENI3054 was transformed in to the Aspergillus niger strain MBin115 (which was prepared as described in WO2004/090155, example 9).

10 transformant were grown in 200 μl YPM in a 96 well microliter dish for 3 days at 34° C.

20 μl of supernatant was run on SDS-PAGE to see if the HSA was expressed by A. niger.

No bands were detectable.

### Example 4

### Cloning of BSA Encoding Gene

BSA has many applications such as media component or for drug delivery.

It is of interest to see if BSA can be expressed at economically interesting levels by the currently used hosts, especially the filamentous fungi.

Bovine liver cDNA library was ordered and received from Stratagene (cat. no. 937712). The following two DNA oligo'es where ordered and received:

150503j6

SEQ ID NO: 11:

GACTCGGGATCCACAATGAAGTGGGTGACTTTTATTTCTC
150503j7

SEQ ID NO: 12:

GACTCGCCGCGGCTCGAGTTAGGCTAAGGCTGTTTGAGTTGA

The following PCR was run using the two DNA oligoes and the cDNA as template along with the Pwo-polymerase (Roche); 94° C. 5 min 25* (94° C. 30 sec. 55° C. 30 sec, 72° C. 3 min) 72° C. 7 min.

The plasmid resulting PCR fragment was cut with BamHI and SacI and the gene was isolated from agarose gel. The plasmid pEN12516 (WO 2004/069872) was cut BamHI and SacI and isolated from agarose gel. The vector and the gene was ligated and transformed into DH10b. The resulting plasmid was named pENI3113.

The gene was sequenced (see sequence at the end).

The cloned BSA has no N-glycosylation site.

### Example 5

### Transformation of BSA cDNA Expression Plasmid into Aspergillus oryzae

The plasmid pENI3113 was transformed in to the Aspergillus oryzae strain JAL355 (WO 2004/069872). This was done as mentioned in WO 2004/069872.

10 transformant were grown in 200 μl YPM in 96 well microtiter dish for 3 days at 34° C.

20 μl of supernatant was run on SDS-PAGE to see if the BSA was expressed by A. oryzae.

No bands were detectable.

### Example 6

### Transformation of BSA cDNA Expression Plasmid into Aspergillus niger

The plasmid pENI3113 was transformed in to the Aspergillus niger strain Mbin15. This was done as mentioned in WO 2004/069872.

10 transformant were grown in 200 μl YPM in 96 well microtiter dish for 3 days at 34° C.

20 μl of supernatant was run on SDS-PAGE to see if the BSA was expressed by A. niger.

No bands were detectable.

### Example 7

### Construction of a HSA Aspergillus Expression Plasmid Based on the Synthetic Gene

The Aspergillus expression plasmid pJaL721 (WO 2003/008575) consist of an expression cassette based on a mutated version the Aspergillus niger neutral amylase 11 promoter and the Aspergillus niger amyloglycosidase terminater (Tamg). Also present on the plasmid is the Aspergillus selective marker amdS from Aspergillus nidulans enabling growth on acetamide as sole nitrogen source and the URA3 marker from Saccharomyces cerevisiae enabling growth of the pyrF defective Escherichia coli strain DB6507 (ATCC 35673).

Transformation into E. coli DB6507 using the S. cerevisiae URA 3 gene as selective marker was done in the following way:

E. coli DB6507 was made competent by the method of Mandel and Higa (Mandel, M. and A. Higa (1970) J. Mol. Biol. 45, 154). Transformants were selected on solid M9 medium (Sambrook et. al (1989) Molecular cloning, a laboratory manual, 2. edition, Cold Spring Harbor Laboratory Press) supplemented with 1 g/l casaminoacids, 500 μg/l thiamine and 10 mg/l kanamycin.

The synthetic HSA gene was cloned into pJaL721 in the following way:

The synthetic gene was purchased from DNA2.0, Menlo Park, Calif., USA (www. Dnatwopointo.com) and received on the plasmid pDrive-G0035R. This plasmid was digested with the restriction enzymes BamH I and Sal I and the 1842 bp fragment harboring the HSA gene was purified from an agarose gel.

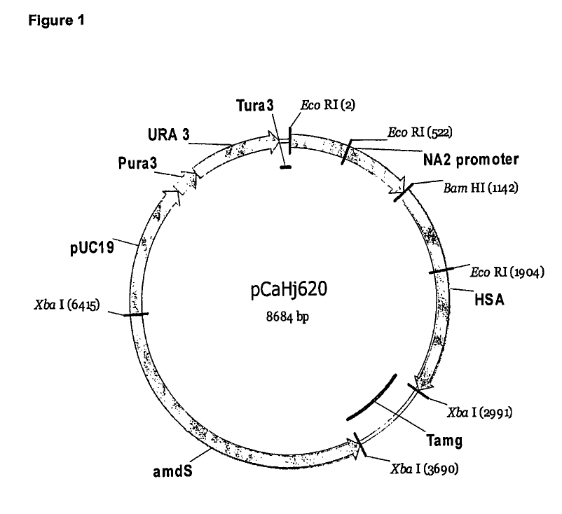

This fragment was ligated to pJaL721 digested with BamH I and Xho I. The ligation mixture was transformed into E. coli DB6507. A plasmid from one of the colonies formed was confirmed to have the expected insert by restriction analysis and DNA sequencing. This plasmid was termed pCaHj620. A restriction map of pCaHj620 is shown in FIG. 1.

### Example 8

### Transformation of the Synthetic HSA Expression Plasmid into Aspergillus oryzae

pCaHj620 was transformed into Aspergillus oryzae BECh2 (WO 00/39322, example 1), a number of Transformants were spore reisolated twice. Spores from second reisolation of each transformant were used to inoculate 10 ml YPM (1% yeast extract, 2% peptone, 2% maltose) in 25 ml NUNC containers. The YPM cultures were grown for 4 days at 34° C. under or bital shaking, and the supernatants were applied to SDS polyacrylamide gels (Bio-Rad, Criterion XT precast gels, Bio-Rad Laboratories, Hercules, Calif., USA) and stained with coomassie blue stain using standard methods. A band of approx. 67 kDal was observed in different amounts from the transformants. The highest yielding transformants were selected for laboratory tank fermentation.

### Example 9

### Transformation of the Synthetic HSA Expression Plasmid into Aspergillus niger

pCaHj620 was transformed into Aspergillus niger MBin118 (WO 2004/090155, Aspergillus niger clean host), a number of Transformants were spore reisolated twice. Spores from second reisolation of each transformant were used to inoculate 10 ml YPM (1% yeast extract, 2% peptone, 2% maltose) in 25 ml NUNC containers. The YPM cultures were grown for 4 days at 34° C. under orbital shaking, and the supernatants were applied to SDS polyacrylamide gels (Bio-Rad, Criterion XT precast gels, Bio-Rad Laboratories, Hercules, Calif., USA) and stained with coomassie blue stain using standard methods. A band of approx. 67 kDal was observed in different amounts from the transformants.

### Example 10

### Construction of a BSA Aspergillus Expression Plasmid Based on the Synthetic Gene

The synthetic BSA gene was cloned into pJaL721 in the following way:

The synthetic gene was purchased from GenScript Corrporation (Scotch Plains, N.J.) (www.genscript.com) and received as a plasmid. This plasmid was digested with the restriction enzymes BamH I and Xho I and the 1837 bp fragment harboring the HSA gene was purified from an agarose gel.

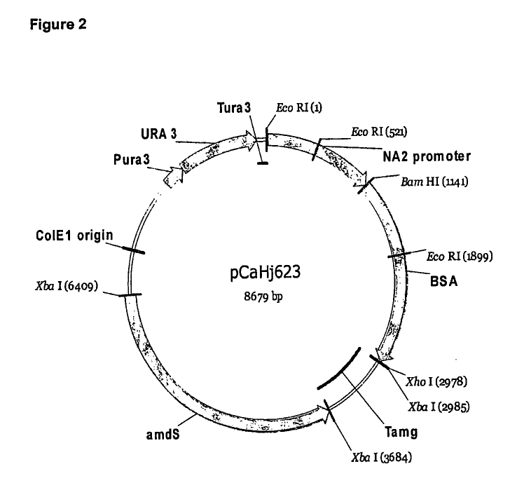

This fragment was ligated to pJaL721 digested with BamH I and Xho I. The ligation mixture was transformed into E. coli DB6507. A plasmid from one of the colonies formed was confirmed to have the expected insert by restriction analysis and DNA sequencing. This plasmid was termed pCaHj623. A restriction map of pCaHj623 is shown in FIG. 2.

### Example 11

### Transformation of the Synthetic BSA Expression Plasmid into Aspergillus oryzae

pCaHj623 was transformed into Aspergillus oryzae BECh2, a number of Transformants were spore reisolated twice. Spores from second reisolation of each transformant were used to inoculate 10 ml YPM (1% yeast extract, 2% peptone, 2% maltose) in 25 ml NUNC containers. The YPM cultures were grown for 4 days at 34° C. under orbital shaking, and the supernatants were applied to SDS polyacrylamide gels (Bio-Rad, Criterion XT precast gels, Bio-Rad Laboratories, Hercules, Calif., USA) and stained with coomassie blue stain using standard methods. A band of approx. 67 kDal was observed in different amounts from the transformants. The highest yielding transformants were selected for laboratory tank fermentation.

### Example 12

### Transformation of the Synthetic BSA Expression Plasmid into Aspergillus niger

pCaHj623 was transformed into Aspergillus niger MBin118, a number of Transformants were spore reisolated twice. Spores from second reisolation of each transformant were used to inoculate 10 ml YPM (1% yeast extract, 2% peptone, 2% maltose) in 25 ml NUNC containers. The YPM cultures were grown for 4 days at 34° C. under orbital shaking, and the supernatants were applied to SDS polyacrylamide gels (Bio-Rad, Criterion XT precast gels, Bio-Rad Laboratories, Hercules, Calif., USA) and stained with coomassie blue stain using standard methods. A band of approx. 67 kDal was observed in different amounts from the transformants.

### Example 13

### Loading of Commercial Fatty Acid Free HSA with Lysed Aspergillus oryzae Cell Extract

Fermentation broth (including cells) from Aspergillus oryzae are treated with ultrasound on ice (4×30 seconds).
Centrifugation @ 4000×g, 5° C., 30 minutes. Supernatant is filtered using 0.22 μm filter.
Ultrafiltration of sample (10 kDa MWCO) overnight in cold room.
On ice, dissolve fatty acid free human serum albumin in the filtered sample, thereby obtaining a concentration of 10 mg HSA/ml.
Add Na-caprylate in 5.4 times molar excess and place the sample in a water bath @ 65° C. When the temperature of the sample reaches 65° C., the incubation is continued for 45 minutes. Then the sample is allowed to cool to room temperature.
Centrifugation @ 4000×g, 5° C., 30 minutes, and filter the supernatant using a 0.45 μm filter.
In a cold room, ultrafiltration of the sample (dilution using Milli-Q water) in order to remove excess Na-caprylate, pigments, etc. (10 kDa MWCO).
Perform buffer-exchange to Dulbecco's Phosphate Buffered Saline+CaCl₂+MgCl₂ (GIBCO™, Ref. 14040-117) by diafiltration (10 kDa MWCO).
Filtration using 0.22 μm filter.

The loaded HSA is now ready for use in serum-free culture medium for primary culture.

### Example 14

### Use of Loaded HSA Compared to Fatty Acid Free HSA

The cell line CHO-K1 was passaged in a growth medium containing nBSA (DMEM:F12 with Ultroser). The cells were then harvested, washed and plated at 500 cells/well in a growth medium without nBSA. Serial dilutions of rHSA obtained from expression in Aspergillus oryzae, and FAF nHSA (from Serologicals) was added to the cells and after 7 days of incubation, cell growth was measured as cell division by incorporation of a radioactively labeled nucleic acid analogue (3H-Thymidine). Results are given as counts Rer minute (cpm). The concentration of HSA in the medium is given in μg/ml. The rHSA obtained by expression in A. oryzae was treated as outlined below before use.

### Sample Preparation:

Germ filtration
Pasteurisation (adding 0.1 g Na-caprylate/L fermentation broth and heating to 65° C. for 1 hour)
Chromatographic capture step
Pool fractions and store @−18° C.
Thaw and filter using 0.22 μm filter
Diafiltration using Milli-Q water and a 10 kDa MWCO filter
Sterile filtration (0.22 μm filter)

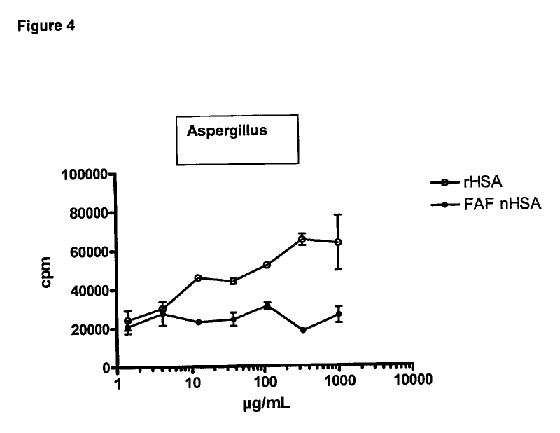

The results are shown in FIG. 4 and indicates that HSA expressed in Aspergillus oryzae (rHSA) is superior to Fatty acid free HSA purified from human serum (FAF nHSA) with regards to the ability to stimulate cell growth. Cpm for cells cultured in the absence of SA was: 11.719. In a second experiment the cell line CHO-K1 was passaged in a growth medium containing nBSA (DMEM:F12 with Ultroser). The cells were then harvested, washed and plated at 10.000 cells/well in a growth medium without nBSA. Serial dilutions of “loaded” and “un-loaded” FAF nHSA (from Serologicals) was added to the cells and after 4 days of incubation, cell growth was measured as cell division, by incorporation of a radioactively labelled nucleic acid analogue (3H-Thymidine). Results are given as counts Rer minute (cpm). The concentration of HSA in the medium is given in μg/ml. Loading of FAF nHSA was performed essentially as outlined in example 13.

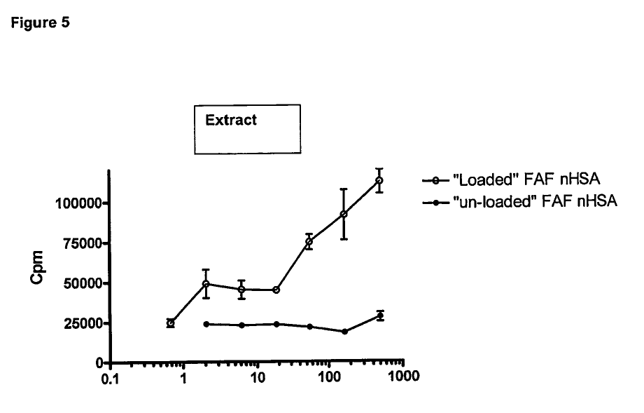

The results are shown in FIG. 5 and clearly indicate that loading of Fatty Acid Free (FAF) nHSA with albumin-free fermentation broth from Aspergillus oryzae, improves the ability of this nHSA to support cell growth. Cpm for cells cultured in the absence of SA was: 25.115.

### Example 15

### Spray-Driving of rHSA

A liquid composition consisting of 3 litre r-HSA solution containing about 97 g/litre was spray dried using a Two-Fluid-Nozzle (TFN) in a Mobile Minor spray dryer from the company Niro A/S, Denmark.

TABLE 2
Drying conditions

| | Inlet | Outlet | Nozzle | |
|---|---|---|---|---|
| Batch no. | temperature | temperature | pressure | Feed rate |
| (Lot. no) | ° C. | ° C. | Bar | g/min. |
| 1 | 120 | 55 | 3.5 | 6.5 |

The powder produced had the characteristics listed in Table 3.

TABLE 3
Powder characteristics

| | | Tapped | Angle of | |
|---|---|---|---|---|
| Bacth no. | Bulk Density | Density | repose | Particle Size |
| (Lot. no) | g/ml | g/ml | degrees | D₅₀ μm |
| 1 | 0.38 | 0.49 | 41 | 6 |

### Example 16

### Spray-Driving Combined with an Integrated Fluid Bed

A liquid feed preparation consisting of 57.7 kg r-HSA solution and 14.4 kg sodium chloride. The feed preparation had a dry matter content of about 28%, where of r-HSA solids constituted ¼ of the total solids.

A Two-Fluid-Nozzle (TFN) was selected for the atomization of the feed. The spray dryer was equipped with an integrated fluid bed attached to the lower part of the conical bottom part of the drying chamber. The spent drying air was removed from the cylindrical drying chamber through the chamber roof. The air with entrained small particles were lead to a cyclone to separate of the entrained particle, so they could get reintroduced into the drying chamber through an annular slit surrounding the atomizing nozzle.

TABLE 4
Drying conditions

| | Inlet | Outlet | Nozzle | |
|---|---|---|---|---|
| Batch no. | temperature | temperature | pressure | Feed rate |
| (Lot. no) | ° C. | ° C. | Bar | kg/g. |
| 3 | 140 | Start: 75 | Start: 3.50 | 30 |
| | | Granulation: 67 | Granulation: 2.25 | |

TABLE 5
Powder characteristics

| | | Tapped | Angle of | |
|---|---|---|---|---|
| Bacth no. | Bulk Density | Density | repose | Particle Size |
| (Lot. no) | g/ml | g/ml | degrees | D₅₀ μm |
| 3 | 0.37 | 0.43 | 34 | 133 |

The particle size was significantly increased due to the changed processing conditions of this example compared to the reference conditions in Example 15. The resulting product was in addition more free-flowing than the reference as the angle of repose was reduced. The bulk and tapped densities were, however, not significantly changed.

It is not simple to quantify if a powder is “easy” or “less easy” to re-constitute into a solvent. For many wide spread products this property is, however, of great importance. The International Dairy Federation (IDF), has developed a standard for measuring wettability, dispersibility and solubility (IDF Standard 087:1979—Determination of the dispersibility & wettability). In Table 6 the results are summarized. The methods are based on IDF Standard # 087 and adopted to rHSA. In all cases the same amount of active r-HSA was used.

TABLE 6
Reconstitution properties of standard spray dried vs. new product

| Batch no. | Wettability | Dispersibility | Solubility |
|---|---|---|---|
| (Lot. no) | Min. | Sec. | % |
| 1 | 8-10 | 120 | 75 |
| 3 | 3 | 50 | 93 |

Wettability: 2 g of new product and 0.5 g of standard product was added to the surface of 100 ml water (22° C.) in a 600 ml beaker. Time for complete dissolution was measured.

Dispersibility: 4 g of new product and 1 g of standard product was added to 100 ml of water (25° C.) in a 600 ml beaker during gentle manual stirring with a spoon. The time for complete dissolution was measured.

Solubility: 10 g of new product and 2.5 g of standard product was added to 100 ml water (25° C.) and stirred with a spoon for 20 sec. A sample of the liquid phase was taken and the dry matter content was measured. The percentage of solids in the solution was calculated.

The results found in Table 6 all show a remarkable improvement of the reconstitution properties of the new product according to this invention.

### Example 17

### Spry-Dried and Agglomerated Liquid Feed Product Comprising HSA

A liquid feed preparation consisting of 100 kg HSA solution containing about 100 g/l HSA and 10 kg or 1 kg or 0.1 kg or 0.0 kg of a salt, preferable sodium chloride, is prepared. The feed preparation is added to a spray drying apparatus of the same set-up as disclosed in Example 16. A product with improved wettability, dispersibility and solubility is obtained when compared to a non-agglomerated dry HSA product.

1. A method for recombinant expression of a wild type serum albumin polypeptide in a filamentous fungal host organism comprising expressing a modified nucleic acid sequence encoding a wild type serum albumin polypeptide in a filamentous fungal host organism, wherein the modified nucleic acid sequence differs in at least one codon from each wild type nucleic acid sequence encoding said wild type serum albumin polypeptide.

2. (canceled)

3. The method according to claim 1, wherein the serum albumin is bovine serum albumin.

4. The method according claim 1, wherein the serum albumin is human serum albumin.

5. The method according to claim 1, wherein the nucleic acid sequences is the sequence of SEQ ID NO: 1 or SEQ ID NO: 3.

6. The method according to claim 1, wherein at least 23, t 50 or 15 codons have been modified.

7. The method according to claim 1, wherein at least 10% 20%, 30%, 50% or 75% of the codons have been modified.

8. The method according to claim 1, wherein the modification of at least one codon results in a codon optimized for translation in the host organism of choice.

9. The method according to claim 7, wherein at least one codon is optimized for translation in a filamentous fungus.

10. The method according to claim 8, wherein codon usage of at least one modified codon corresponds to the codon usage of alpha amylase from Aspergillus oryzae.

11. The method according to claim 1, wherein the filamentous fungal host organism is selected from the group consisting of Acremonium, Aspergillus, Fusarium, Humicola, Mucor, Myceliophthora, Neurospora, Penicillium, Thielavia, Tolypocladium, and Trichoderma.

12. The method according to claim 11, wherein the Aspergillus cell is Aspergillus awamori, Aspergillus foetidus, Aspergillus japonicus, Aspergillus niger, Aspergillus nidulans, or Aspergillus oryzae.

13. The method according to claim 11, wherein the host cell is A. oryzae or A. niger.

14. The method according to claim 7, wherein the serum albumin protein is encoded by a nucleic acid sequence codon optimized in at least 10%, 20%, 30%, 50% or 75% of the codons.

15. The method according to claim 1, wherein the modified nucleic acid sequences encoding BSA or HSA are selected from the group consisting of SEQ ID NO: 5 and SEQ ID NO: 7.

16. A modified nucleic acid sequence encoding a wild type bovine serum albumin polypeptide and capable of expression in a filamentous fungal host organism, wherein said modified nucleic acid sequence differs in at least one codon from each wild type nucleic acid sequence encoding said wild type bovine serum albumin polypeptide.

17. (canceled)

18. (canceled)

19. (canceled)

20. The modified nucleic acid sequence according to claim 16, wherein the modification of at least one codon results in a codon optimized for translation in Aspergillus sp.

21. The modified nucleic acid sequence according to claim 16, wherein the codon usage corresponds to the codon usage of alpha amylase from Aspergillus oryzae.

22. The modified nucleic acid sequence according to claim 20, wherein the Aspergillus is Aspergillus awamori, Aspergillus foetidus, Aspergillus japonicus, Aspergillus niger, Aspergillus nidulans, or Aspergillus oryzae.

23. The modified nucleic acid sequence according to claim 22, wherein the Aspergillus is A. oryzae or A. niger.

24. (canceled)

25. (canceled)

26. (canceled)

27. (canceled)

28. A loaded serum albumin obtainable by,
i) recombinant expression of a nucleic acid sequence encoding the serum albumin in a filamentous fungal host cell; and/or
ii) loading the serum albumin by contacting said serum albumin with a cell extract derived from filamentous fungal cells.

29. The loaded serum albumin according to claim 28, wherein the filamentous fungal host cell is selected from the group consisting of Acremonium, Aspergillus, Fusarium, Humicola, Mucor, Myceliphthora, Neurospora, Penicillium, Thielavia, Tolypocladium, or Trichoderma.

30. The loaded serum albumin according to claim 29, wherein the Aspergillus cell is Aspergillus awamori, Aspergillus foetidus, Aspergillus japonicus, Aspergillus niger, Aspergillus nidulans, or Aspergillus oryzae.

31. The loaded serum albumin according to claim 28, wherein the serum albumin is HAS or BSA.

32. The loaded serum albumin according to claim 28, wherein the nucleic acid sequence is selected from the group consisting of SEQ ID NO: 5 and SEQ ID NO: 7.

33. The loaded serum albumin according to claim 28, wherein the cell extract is obtained by lysing the filamentous fungal host cells.

34-52. (canceled)
